(19) United States
(12) Patent Application Publication
(10) Pub. No.: US 2022/0079966 A1
(43) Pub. Date: Mar. 17, 2022

(54) HYALURONAN SYNTHESIS INHIBITION FOR TREATING AUTOIMMUNE, INFLAMMATORY, FIBROTIC, OR PROLIFERATIVE DISEASES OR DISORDERS

(71) Applicant: The Board of Trustees of the Leland Stanford Junior University, Stanford, CA (US)

(72) Inventors: Paul L. Bollyky, Stanford, CA (US); Nadine Nagy, Stanford, CA (US); Gernot Kaber, Palo Alto, CA (US)

(21) Appl. No.: 17/415,647

(22) PCT Filed: Dec. 20, 2019
(86) PCT No.: PCT/US2019/067911

§ 371 Date: Jun. 17, 2021

Related U.S. Application Data
(60) Provisional application No. 62/783,020, filed on Dec. 20, 2018.

(51) Int. Cl. A61K 31/7048; A61P 35/00; A61P 3/10; A61P 37/06

(52) U.S. Cl. CPC A61K 31/7048; A61P 37/06; A61P 3/10; A61P 35/00

(57) ABSTRACT

Compositions for treating an autoimmune, inflammatory, fibrotic, or proliferative disease or disorder comprising a compound that inhibits hyaluronan synthesis and a pharmaceutically acceptable carrier are described. In some embodiments, the compound that inhibits hyaluronan synthesis is 4-methylumbelliferone-glucuronide. Methods for treating an autoimmune, inflammatory, fibrotic, or proliferative disease or disorder, including administering to the subject a composition having a compound in an amount effective to inhibit hyaluronan synthesis in a mammalian subject, are also described.

### STATEMENT OF GOVERNMENT LICENSE RIGHTS

This invention was made with Government support under contracts AI101984 and DK096087 awarded by the National Institutes of Health. The Government has certain rights in this invention.

### BACKGROUND

Hyaluronan (HA) is an extracellular matrix glycosaminoglycan with many roles in normal tissue function and development (Fraser J. R., et al., J. Intern. Med. 1997; 242(1):27-33; Jiang D, et al., Physiol. Rev. 2011; 91(1):221-264; Termeer C., et al., Trends Immunol. 2003; 24(3):112-114). HA is synthesized by three hyaluronan synthase (HAS) enzymes, HAS1, HAS2, and HAS3 (Weigel P. H., et al., J. Biol. Chem. 2007; 282(51):36777-36781). These enzymes lengthen HA by repeatedly adding glucuronic acid (GlcUA) and N-acetyl-glucosamine (GlcNAc) to the nascent polysaccharide as it is extruded through the cell membrane into the extracellular space (Weigel P. H., et al., J. Biol. Chem. 2007; 282(51):36777-36781).

There is substantial experimental and therapeutic interest in inhibiting HA synthesis. HA is known to promote inflammatory responses (Jiang D., et al., Physiol. Rev. 2011; 91(1):221-264) including the activation and maturation of multiple immune cell types (Termeer C., et al., J. Exp. Med. 2002; 195(1):99-111), the release of pro-inflammatory chemokines and cytokines (Taylor K. R., et al., J. Biol. Chem. 2004; 279(17):17079-17084; McKee C. M., et al., J. Clin. Invest. 1996; 98(10):2403-2413) and proliferation (Mahaffey C. L., et al., J. Immunol. 2007; 179(12):8191-8199) and migration (Itano N., et al., Proc. Natl. Acad. Sci. USA 2002; 99(6):3609-3614) of leukocytes. HA and its receptor interactions are also known to influence both the number and function of lymphocytes (Bollyky P. L., et al., J. Immunol. 2009; 183(4):2232-2241; Bollyky P. L., et al., J. Immunol. 2007; 179(2):744-747; Bollyky P. L., et al. Proc. Natl. Acad. Sci. USA 2011; 108(19):7938-7943). HA levels are greatly elevated in chronically inflamed tissues (Cheng G., et al., Matrix Biol. 2011; 30(2):126-134; Kang L., et al., Diabetes 2013; 62(6):1888-1896; Mine S., et al., Endocr. J. 2006; 53(6):761-766) including in the tumor microenvironment, in fibrosis, and at sites of autoimmunity (Bollyky P. L., et al., Curr. Diab. Rep. 2012; 12(5):471-480; Nagy N., et al., J Clin Invest. 2015; 125(10):3928-3940). In autoimmune type 1 diabetes (T1D) HA accumulates in pancreatic islets (Bogdani, M., et al., Curr. Diab. Rep. 2014; 14:552; Bogdani, M., et al., Diabetes 2014; 63:2727-2743). In obesity-associated type 2 diabetes (T2D), HA accumulates within inflamed tissues in response to hyperglycemia (Shakya, S., et al., Int. J. Cell Biol. 2015:701738; Wang, A., et al., Autophagy 5:864-865), inflammatory cytokines (Bollyky, P. L., et al., Cell. Mol. Immunol. 7:211-220), and other triggers (Jiang, D., et al., Physiol. Rev. 91:221-264; Laurent, T. C., et al., Immunol. Cell Biol. 74:A1-7; Lauer, M. E., et al., J. Biol. Chem. 284:5299-5312). HA is increased in skeletal muscle (Kang L, et al., Diabetes 2013; 62(6):1888-1896) and adipose tissue (Liu, L. F., et al., Diabetologia 2015; 58:1579-1586) in T2D. HA is also increased in setting of chronic inflammation and fibrosis, including liver cirrhosis, primary sclerosing cholangitis, kidney fibrosis and other fibrotic conditions (Lewis, A., et al., Histol. Histopathol. 2008; 23:731-739; Li, Y., et al., J. Exp. Med. 2011; 208:1459-1471; Orasan, O. H., et al., Clujul Med 2016; 89:24-31; Colombaro, V., et al., Nephrol. Dial. Transplant. 2013; 28:2484-2493; Vesterhus, M., et al., Hepatology 2015; 62(1):188-197).

Increases in HA are associated with many chronic disease processes with unremitting inflammation, including type 2 diabetes, (Mine, S., et al., Endocr. J. 2006; 53:761-766; Kang, L. et al., 2013; Diabetes 62:1888-1896), liver cirrhosis (Plevris, J. N., et al., Eur. J. Gastroenterol. Hepatol. 2000; 12(10):1121-1127), asthma, and other chronic inflammatory diseases of diverse etiologies (Plevris, J. N. et al., 2000; Eur. J. Gastroenterol. Hepatol. 12:1121-1127; Wells, A. F. et al., Transplantation 1990; 50:240-243; Dahl, L. B., et al., Ann. Rheum. Dis. 1985; 44:817-822; Hallgren, R., et al., Am. Rev. Respir. Dis. 1989; 139:682-687; Evanko, S. P., et al., Am. J. Pathol. 1998; 152:533-546); Cheng, G. et al., Matrix Biol. 2011; 30:126-134; Ayars, A. G. et al., Int. Arch. Allergy Immunol. 2013; 161:65-73; Liang, J. et al., J. Allergy Clin. Immunol. 2011; 128:403-411.e3).

HA has been implicated in multiple autoimmune diseases including rheumatoid arthritis (Yoshioka, Y., et al., 2013; Arthritis Rheum. 65(5):1160-1170), lupus (Yung S., et al., Autoimmune Dis. 2012; 2012:207190, PMID:22900150), Sjogren's syndrome (Tishler M., et al., Ann Rheum Dis. 1998; 57(8):506-508), and Hashimoto's thyroiditis (Gianoukakis, A. G., et al., Endocrinology. 2007; 148(1):54-62). HA surrounds tumors in diverse forms of cancer (Toole, B. P. Nat. Rev. Cancer 2004; 4:528-539). This accumulation of HA is part of a larger pattern of ECM deposition associated with persistent inflammation. HA increases local edema (Waldenstrom, A., et al., J. Clin. Invest. 1991; 88:1622-1628) and contributes to an inflammatory cascade that drives leukocyte migration, proliferation, differentiation through effects on gene expression and cytokine production and cell survival. These pathways and the impact of HA production on innate immunity are the subject of several reviews (Jiang, D., et al., Annu. Rev. Cell Dev. Biol. 2007; 23:435-461. PMID: 17506690); Jiang, D., et al., Physiol. Rev. 2011; 91:221-264; Petrey, A. C. et al., Front. Immunol. 2014; 5:101; Slevin, M. et al., Matrix Biol. 2007; 26:58-68; Sorokin, L. Nat. Rev. Immunol. 2010; 10:712-723).

Increased HA is found in many cancers and is important for cancer progression and metastasis (Schwertfeger, K. L., et al., Front. Immunol. 2015; 6:236; Misra, S., et al., FEBS J. 2011; 278:1429-1443; Li, Y., et al., Br. J. Cancer 2001; 85:600-607). HA has been implicated in the pathogenesis of many cancers, for example, pancreatic cancer (Nakazawa H, et al., Cancer Chemother. Pharmacol. 2006; 57:165-170; Morohashi H, et al., Biochem. Biophys. Res. Comm. 2006; 345:1454-1459; Hajime M, et cd., int. J. Cancer 2007; 120:2704-2709), prostate cancer (Lokeshwar V B, et al., Cancer Research 2010; 70:2613-2623), skin cancer (Kudo D, et al., Biochem. Biophys. Res. Comm. 2004; 321:783-787; Bhattacharyya, S. et al., Eur. J. Pharmacol. 2009; 614:128-136; Edward M., et al., Br. J. Dermatol. 2010; 162:1224-1232), esophageal cancer, breast cancer (Urakawa H., et al., Int. J. Cancer 2012; 130:454-466; Saito T., et al., Oncol. Rep. 2013; 29:335-342; Saito T., el al., Oncol. Lett. 2013; 5:1068-1074), liver cancer (Kundu B., et al., Biomaterials (2013) 34:9462-9474), bone cancer/metastases (Okuda H., et al., Cancer Research 2012; 72:537-547), leukemia (Lompardía S. L., et al., Glycobiology 2013; 23:1463-1476), endometrial, stomach, testes, thyroid, cervical, esophageal, and ovarian cancer (Tamura R., et al., J. Ovarian Res. 2014; 7:94).

Numerous studies implicate HA as a driving factor in inflammation and HA is implicated in a wide range of inflammatory disorders (Hull R. L., et al., J. Histochem. Cytochem. 2012; 60(10):749-760; Kuipers H. F., et al., Proc. Natl. Acad. Sci. USA 2016; 113(5):1339-44; Yoshioka Y., et al., Arthritis Rheum. 2013; 65(5):1160-1170). These include, for example, renal ischaemia-reperfusion injury (Colombaro V., et al., Nephrol. Dial. Transplant. 2013; 28:2484-2493), asthma (Liang, J., et al., J. Allergy Clin. Immunol. 2011; 128(2):403-411.e3, 2011; Forteza R. M., et al., J. Biol. Chem. 2012; 287:42288-42298), pulmonary hypertension (Collum S. D., et al., Br. J. Pharmacol. 2017; 174(19):3284-3301), obesity-associated type 2 diabetes (Sim M.-O., et al., Chem. Biol. Interact. 2014; 216:9-16), arthritis (Campo, G. M., et al., BioFactors 2012; 38(1):69-76), atherosclerosis (Fischer, J. W., Matrix Biol. 2019; 78-79:324-336), wound healing (David-Raoudi, M., et al., Wound Repair Regen. 2008; 16(2):274-287), chronic obstructive pulmonary disease and emphysema (Dentener M. A., et al., Thorax 2005; 60(2):114-119), bronchiolitis obliterans syndrome (BOS) (Todd J. L., et al., Am. J. Respir. Crit. Care Med. 2014; 189(5):556-566), transplant rejection (Tesar B. M., et al., Am. J. Transplant. 2006; 6(11):2622-2635), graft versus host disease, dermatomyositis (Kubo M., et al., Arch. Dermatol. Res. 1998; 290(10):579-581), and inflammatory bowel disease (de la Motte C. A., et al., Int. J. Cell Biol. 2015; 2015:481301).

Numerous studies implicate HA as a driving factor in fibrosis and HA is implicated in a wide range of fibrotic disorders. These include liver fibrosis (Halfon, P., et al., Comp. Hepatology 2005; 4:6), renal fibrosis (Kato, N., et al., Am. J. Pathology 2011; 178(2):572-579), dermal fibrosis (Tolg, C., et al., Am. J. Pathology 2012; 181(4):1250-1270) intestinal fibrosis (Rieder, F., et al., Nature Reviews Gastroenterology and Hepatology 2009; 6(4):228-235), and lung fibrosis (Bensadoun, E. S., et al., Am. J. Respir. Critical Care Medicine 1996; 154(6):1819-1828); Venkatesan, N., et al., Am. J. Respir. Critical Care Medicine 2000; 161(6):2066-2073).

HA-mediated inflammatory signals can be particularly relevant in settings of sterile inflammation such as cancer, fibrosis, inflammation, and autoimmunity (Taylor K. R., et al. J. Biol. Chem. 2007; 282(25):18265-18267). At most sites of injury, HA is rapidly cleared. However, at sites of chronic inflammation, HA persists (Bollyky P. L., et al., J. Leukoc. Biol. 2009; 86(3):567-572). This can have important consequences for local immune regulation (Bollyky P. L., et al., Curr. Diab. Rep. 2012; 12(5):471-480; Garantziotis S., et al., Am. J. Respir. Crit. Care Med. 2010; 181(7):666-675; Hull R. L., et al., J. Histochem. Cytochem. 2015; 63(8):592-603; Yung S., et al., Autoimmune Dis. 2012; 2012:207190; Lee-Sayer S. S., et al., Front. Immunol. 2015; 6:150; Jackson D. G. Immunol. Rev. 2009; 230(1):216-231; Petrey A. C., et al., Front. Immunol. 2014; 5:101).

HA is produced by three synthases, HAS1, HAS2, and HAS3, and is abundant at sites of chronic inflammation. Catabolic, low-molecular weight fragments of HA (LMW-HA) act as endogenous danger signals that promote antigenic responses (Termeer, C. et al. J. Exp. Med. 2002; 195:99-111) and immune activation (Jiang, D., et al., Physiol. Rev. 2011; 91:221-264) via CD44 and Toll-like receptor (TLR) signaling (Jiang, D. et al., Nat. Med. 2005; 11:1173-1179; Fieber, C. et al., J. Cell. Sci. 2004; 117:359-367; Termeer, C., et al., Trends Immunol. 2003; 24:112-114 (2003); Taylor, K. R. et al., J. Biol. Chem. 2004; 279:17079-17084).

LMW-HA also promotes the activation and maturation of dendritic cells (DC) (Termeer, C. et al., J. Exp. Med. 2002; 195:99-111), drives the release of pro-inflammatory cytokines such as IL-1, TNF-alpha, IL-6 and IL-12 by multiple cell types (Bollyky, P. L. et al., J. Immunol. 2007; 179:744-747; Bollyky, P. L. et al., Proc. Natl. Acad. Sci. USA 2011; 108:7938-7943), drives chemokine expression and cell trafficking (McKee, C. M. et al., J. Clin. Invest. 1996; 98:2403-2413), and promotes proliferation (Scheibner, K. A. et al., J. Immunol. 2006; 177:1272-1281) and angiogenesis (Kuipers, H. F. et al., Proc. Natl. Acad. Sci. USA 2016; 113:1339-1344).

It was recently reported that HA deposits accumulate within the pancreatic islets of individuals with recent-onset T1D and these deposits were present at sites of insulitis (Bogdani, M. et al., Diabetes 2014; 63:2727-2743). Similar HA deposits were observed in animal models of type 1 diabetes (Nagy, N. et al., J. Clin. Invest. 2015; 125(10):3928-3940). This HA consists of catabolic, fragments of low molecular weight HA (LMW-HA). Because HA overexpression and HA fragments in particular are known to drive inflammation (Olsson, M., et al., PLoS Genet. 2011; 7(3):e1001332); Yoshioka, Y. et al., Arthritis Rheum. 2013; 65:1160-1170), and without wishing to be bound by theory, it is possible that HA drives the pathogenesis of type 1 diabetes.

4-methylumbelliferone (4-MU) is a small molecule inhibitor of HA synthesis (Nagy N., et al., Front. Immunol. 2015; 6:123). 4-MU inhibits HA production in multiple cell lines and tissue types both in vitro and in vivo (Yoshioka Y., et al., Arthritis Rheum. 2013; 65(5):1160-1170; Bollyky P. L., et al., Cell Mol. Immunol. 2010; 7(3):211-220; Nagy N., et al., Circulation 2010; 122(22):2313-2322).

4-MU is thought to inhibit HA production in at least two ways. First, 4-MU is thought to function as a competitive substrate for UDP-glucuronyltransferase (UGT), an enzyme involved in HA synthesis (Kakizaki, I., et al., J. Biol. Chem. 2004; 279(32):33281-9; PMID:15190064). HA is produced by the HA synthases HAS1, HAS2 and HAS3 from the precursors UDP-glucuronic acid (UDP-GlcUA) and UDP-N-acetyl-glucosamine (UDP-GlcNAc). These are generated by the transfer of a UDP residue to N-acetylglucosamine and glucuronic acid via the UDP-glucuronyltransferase (UGT). The availability of UDP-GlcUA and UDP-GlcNAc thereby control HA synthesis (Vigetti, D., et al., J. Biol. Chem. 2012; 287(42):35544-35555). In the presence of 4-MU, it covalently binds through its hydroyxl group at position 4 to glucuronic acid via the UGT. As a consequence, the concentration of UDP-glucuronic acid declines in the cytosol and HA synthesis is reduced. This therewith reduces 4-MU the UDP-GlcUA content inside the cells. 4-MU inhibits HA synthesis by depleting the HAS enzyme UDP-GlcUA, which is consumed by 4-MU glucuronidation. So far it is unclear how exactly the second mechanism works, but, 4-MU reduces expression of HAS mRNA expression (Kultti, A., et al., Exp. Cell Res. 2009; 315(11):1914-1923) as well as mRNA for UDP glucose pyrophosphorylase and dehydrogenase (Saito, T., et al., Oncol. Lett. 2013; 5 (3): 1068-1074).

4-MU treatment prevents many of the inflammatory phenotypes associated with HA, including tumor metastasis, fibrosis progression and autoimmunity (reviewed in (Nagy N., et al., Front. Immunol. 2015; 6:123)). It has been previously reported that 4-MU promotes the induction of Foxp3+ regulatory T-cells, an important anti-inflammatory cell type, and that 4-MU prevents fibrosis and autoimmunity in multiple animal models of human autoimmune diseases, including multiple sclerosis, T1D, and rheumatoid arthritis (Nagy N., et al., J. Clin. Invest. 2015; 125(10):3928-40; Kuipers H. F., et al., Proc. Natl. Acad. Sci. USA 2016; 113(5):1339-1344; Yoshioka Y., et al., Arthritis Rheum. 2013; 65(5):1160-1170; Nagy N., et al., Front. Immunol. 2015; 6:123; Mueller A. M., et al., J. Biol. Chem. 2014; 289(33):22888-22899).

A few studies have investigated the impact of 4-MU on HA synthesis in autoimmunity and inflammation. In vivo studies showed that 4-MU treatment prevented lung injury and reduced inflammatory cytokine levels in mouse models of staphylococcal enterotoxin-mediated (McKallip, R. J., et al., Toxins (Basel). 2013; 5 (10): 1814-1826) and lipopolysaccharide-mediated acute lung injury (McKallip, R. J., et al., Inflammation 2015; 38:1250-1259). 4-MU has also been shown to have protective effects on non-infectious inflammation, including renal ischemia and reperfusion (Colombaro, V. et al., Nephrol. Dial. Transplant. 2013; 28:2484-2493), and airway inflammation secondary to cigarette smoke (Forteza, R. M., et al., J. Biol. Chem. 2012; 287(50):42288-42298). 4-MU also restores normoglycemia and promotes insulin sensitivity in obese, diabetic mice via increased production of adiponectin (Sim, M.-O., et al., Chem. Biol. Interact. 2014; 216:916). 4-MU has also been reported to ameliorate disease in a limited number of mouse models of autoimmune disease. Specifically, 4-MU treatment was beneficial in the collagen-induced arthritis model where it improved disease scores and reduced expression of matrix metalloproteases (MMPs) (Yoshioka, Y., et al., 2013; 65(5):1160-1170, PMID:23335273).

More recently, 4-MU treatment was demonstrated to prevent and treat disease in the experimental autoimmune encephalomyelitis (EAE) model where it increased populations of regulatory T-cells and polarized T-cell differentiation away from pathogenic, T-helper 1 T-cell subsets and towards non-pathogenic T-helper 2 subsets (Mueller, A. M., et al., J. Biol. Chem. 2014; 289:22888-22899). In addition, 4-MU treatment reduced the number of tumor satellites (Piccioni, F., et al., Glycobiology. 2012; 22(3):400-410), inhibited angiogenesis and cell growth in tumors (Garcia-Vilas, J. A., et al., J. Agric. Food Chem. 2013; 61(17):4063-4071). The existing in vitro and in vivo data suggest that hymecromone may have utility as a component of therapeutic regimens directed against HA-producing cancers. It has been reported that 4-MU treatment prevented cell-cell interactions required for antigen presentation (Bollyky, P. L. et al., Cell. Mol. Immunol. 2010; 7:211-220) and others have described inhibitory effects on T-cell proliferation (McKallip, R. J., et al., Toxins (Basel) 2013; 5(10): 1814-1826). These effects are consistent with established roles for HA and its receptors in T-cell proliferation, activation, and differentiation (Jiang, D., et al., Physiol. Rev. 2011; 91:221-264; Guan, H., et al., J. Immunol. 2009; 183:172-180; Ponta, H., et al., Nat. Rev. Mol. Cell Biol. 2003; 4:33-45). There are also indications that 4-MU treatment may make some models of inflammation worse. 4-MU treatment was associated with worse atherosclerosis in ApoE-deficient mice fed a high-fat diet (Nagy, N. et al., Circulation 2010; 122:2313-2322).

It has been reported that 4-MU treatment limits the progression of EAE (Mueller, A. M., et al., J. Biol. Chem. 2014; 289(33):22888-22899; Kuipers et al., Proc. Natl. Acad. Sci. USA. 2016; 113(5):1339-1344) and autoimmune diabetes in both the DORmO and NOD mouse models (Nagy, N., et al., J. Clin. Invest. 2015; 125(10):3928-3940; PMID:26368307; Kuipers, H. F., et al., Clin. Exp. Immunol. 2016; 185:372-381). This therapeutic effect is shown to be not only a result of the polarization of the T cell response away from a pathogenic Th1 response, but also the reduction of infiltration of these cells into sites of autoimmune attack. Additionally, because 4-MU treatment lifts the inhibition of Foxp3+ Treg induction and function by LMW-HA, this inhibition of the pathogenic response is aided by an increase of Treg numbers (Kuipers, H. F., et al., Clin. Exp. Immunol. 2016; 185:372-381; Mueller, A. M., et al., J. Biol. Chem. 2014; 289(33):22888-22899; Nagy, N., et al., J. Clin. Invest. 2015; 125(10):3928-3940; PMID:26368307). Furthermore, in addition to sustaining a pro-inflammatory environment in MS lesions, HA deposits have been show to inhibit the maturation of oligodendrocytes, the myelin forming cells of the CNS, in MS and other myelin degenerative disorders, and as such are thought to prevent repair of myelin, further contributing to MS pathogenesis (Back, S. A., et al., Nat. Med. 2005; 11(9):966-972; Sloane, J. A., et al., Proc. Natl. Acad. Sci. USA 2010; 107(25):11555-11560; Preston, M., et al., Ann. Neurol. 2013; 73(2):266-280; Bugiani, M., et al., Brain 2013; 136(Pt 1):209-322).

Without wishing to be bound by theory, it is possible that 4-MU treatment can restore the HA load in inflamed tissues to a dominance of anti-inflammatory HMW polymers. Thus, there is great interest in identifying pharmacologic tools to inhibit HA synthesis.

4-MU is a commercially available drug approved for use in humans. Called “Hymecromone” it is prescribed in European and Asian countries to prevent biliary spasm (Takeda S, et al., J. Pharmacobiodyn. 1981; 4(9):724-734). This suggests that 4-MU could be repurposed to inhibit HA synthesis in humans. Indeed, 4-MU is under investigation in human clinical trials as a treatment for HA-associated fibrotic liver and autoimmune biliary diseases (ClinicalTrials.gov Identifiers: NCT00225537, NCT02780752).

Unfortunately, 4-MU has poor pharmacokinetics thought to limit its use outside the biliary tract. The systemic oral bioavailability of 4-MU is reported to be <3%, mostly due to extensive first pass glucuronidation in the liver and small intestine (Garrett E. R., et al., Biopharm. Drug Dispos. 1993; 14(1):13-39; Kultti A. L., et al., Exp. Cell. Res. 2009; 315(11):1914-1923). Any 4-MU that does reach the systemic circulation is rapidly metabolized with a half-life of 28 minutes in humans (3 minutes in mice) and <1% of a given dose is excreted unchanged in the urine (Garrett E. R., et al., Biopharm. Drug Dispos. 1993; 14(1):13-39; Kultti A., et al., Exp. Cell. Res. 2009; 315(11):1914-1923). Consequently, the median plasma concentration of 4-methlyumbelliferyl glucuronide (4-MUG) is more than 3,000 fold higher than that of 4-MU in mouse models (Nagy N., et al., Front. Immunol. 2015; 6:123). Analogous findings have been reported in healthy human volunteers (Garrett E. R., et al., Biopharm. Drug Dispos. 1993; 14(1):13-39). Despite poor bioavailability and a short half-life, oral administration of 4-MU nonetheless inhibits HA synthesis in vivo, suggesting additional factors may potentiate its activity and sustained effect.

It has been discovered that 4-MUG is biologically active and directly inhibits HA synthesis. This was not expected or obvious, particularly since most glucuronide metabolites are not bioactive. As described herein, the inventors have shown that that 4-MUG can inhibit HA synthesis in a variety of contexts, including in cancer, autoimmunity, fibrotic and inflammatory diseases.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In one aspect, the present disclosure provides a composition for treating an autoimmune, inflammatory, fibrotic, or proliferative disease or disorder comprising (i) a compound that inhibits hyaluronan synthesis, and (ii) a pharmaceutically acceptable carrier.

In one embodiment, the compound is a UDP-glycosyltransferase inhibitor.

In one embodiment, the compound is a UDP-glucuronyltransferase inhibitor.

In one embodiment, the compound is 4-methylumbelliferone-glucuronide.

In one embodiment, the compound is effective to induce a regulatory T-cell response.

In one embodiment, the compound is effective to increase FoxP3+ regulatory T-cells.

In one embodiment, the autoimmune disease or disorder is selected from the group consisting of amyloidosis, ankylosing spondylitis, nephritis, antiphospholipid syndrome, autoimmune angioedema, autoimmune encephalomyelitis, autoimmune hepatitis, autoimmune orchitis, autoimmune pancreatitis, autoimmune retinopathy, autoimmune urticaria, Behcet's disease, benign mucosal pemphigoid, bullous pemphigoid, celiac disease, Chagas disease, CREST syndrome, Crohn's disease, fibromyalgia, Graves' disease, Guillain-Barre syndrome, Hashimoto's thyroiditis, hemolytic anemia, Henoch-Schonlein purpura (HSP), nephropathy, juvenile arthritis, juvenile diabetes (Type 1 diabetes), lupus, multiple sclerosis, neuromyelitis optica, polyarteritis nodosa, primary biliary cirrhosis, primary sclerosing cholangitis, psoriasis, psoriatic arthritis, rheumatoid arthritis, sarcoidosis, scleroderma, Sjogren's syndrome, temporal arteritis (giant cell arteritis), ulcerative colitis (UC), vasculitis, and vitiligo.

In one embodiment, the inflammatory disease or disorder is selected from the group consisting renal ischaemia-reperfusion injury, asthma, pulmonary hypertension, type 2 diabetes, arthritis, atherosclerosis, wound healing, chronic obstructive pulmonary disease (COPD), emphysema, bronchiolitis obliterans syndrome (BOS), allogeneic transplant rejection, graft versus host disease, dermatomyositis, inflammatory bowel disease, and stroke. In another embodiment, the inflammatory disease or disorder is type 2 diabetes, allogenic transplant rejection, or graft versus host disease.

In one embodiment, the fibrotic disease or disorder is selected from the group consisting of primary sclerosing cholangitis, biliary cirrhosis, biliary spasm, cirrhosis, liver fibrosis, renal fibrosis, dermal fibrosis, intestinal fibrosis, and lung fibrosis.

In one embodiment, the proliferative disease or disorder is selected from the group consisting of pancreatic cancer, prostate cancer, skin cancer, esophageal cancer, breast cancer, liver cancer, bone cancer, ovarian cancer, kidney cancer, anal cancer, brain cancer, biliary cancer, melanoma, insulinoma, endometrial cancer, stomach cancer, testes cancer, thyroid cancer, cervical cancer, and lymphoma. In another embodiment, the proliferative disease or disorder is melanoma, insulinoma, lymphoma, or ovarian cancer.

In one aspect, the present disclosure provides a method for treating an autoimmune, inflammatory, fibrotic, or proliferative disease or disorder in a mammalian subject in need thereof, the method comprising administering to the subject a composition comprising a compound in an amount effective to inhibit hyaluronan synthesis in the mammalian subject.

In one embodiment, the compound is a UDP-glycosyltransferase inhibitor.

In one embodiment, the compound is a UDP-glucuronyltransferase inhibitor.

In one embodiment, the compound is 4-methylumbelliferone-glucuronide.

In one embodiment, the compound is effective to induce a regulatory T-cell response.

In one embodiment, the compound is effective to increase FoxP3+ regulatory T-cells.

In one embodiment, the mammalian subject is a human subject.

In one aspect, the present disclosure provides a method for treating a proliferative disease and/or reversing progression of a proliferative disease in a mammalian subject suffering from or at risk of developing a proliferative disease comprising administering to the mammalian subject a composition comprising a compound in an amount effective to inhibit hyaluronan synthesis in the mammalian subject.

In one embodiment, the compound is a UDP glycosyltransferase inhibitor or a UDP glucuronyltransferase inhibitor.

In one embodiment, the compound is 4 methylumbelliferone-glucuronide.

In one embodiment, the mammalian subject is a human subject.

In one embodiment, the proliferative disease is melanoma, insulinoma, lymphoma, ovarian cancer.

In one aspect, the present disclosure provides a method for treating type 1 diabetes or type 2 diabetes in a mammalian subject in need thereof, the method comprising administering to the subject a composition comprising a compound in an amount effective to inhibit hyaluronan synthesis in the mammalian subject.

In one embodiment, the compound is a UDP glycosyltransferase inhibitor or a UDP glucuronyltransferase inhibitor.

In one embodiment, the compound is 4 methylumbelliferone-glucuronide.

In one embodiment, the mammalian subject is a human subject.

In one embodiment, the compound is effective to induce a regulatory T-cell response.

In one embodiment, the compound is effective to increase FoxP3+ regulatory T-cells.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1A illustrates molecular structures for 4-MU and its primary metabolites, 4-MUG and 4-MUS.

FIG. 1B shows concentrations of 4-MU and its metabolites in plasma of animals fed 4-MU chow for two weeks, measured via HPLC.

FIG. 1C shows different concentrations of 4-MU and 4-MUG in the serum of mice fed 4-MU for two weeks measured via HPLC.

FIG. 1D shows HA production by B16F10 cells cultured for 48 hours in 4-MU.

FIG. 1E shows HA production by B16F10 cells cultured for 48 hours in 4-MUG.

FIG. 1F shows representative images of HA staining in B16F10 cells cultured in DMSO as control (left), 4-MU (middle) or 4-MUG (right).

FIG. 1G shows HA synthesis inhibition upon treatment with 4-MU or 4-MUG in CTLL2 cells.

FIG. 1H shows HA synthesis inhibition upon treatment with 4-MU or 4-MUG in Min6 cells.

FIG. 2A shows fluorescence visualization in wells of a 96-well plate which was filled with 200 μl PBS and 10% FCS, in some wells 4-MU (middle) and 4-MUG (right) were added, control wells remained untreated (left).

FIG. 2B shows fluorescent signal over time measured as mean fluorescent intensity (MFI) after 4-MU and 4-MUG were separately added to DMEM. Fluorescent values of 4-MUG were normalized to the 4-MU fluorescence.

FIG. 2C shows fluorescence of 4-MU and 4-MUG from B16F10 cells incubated for 24, 48, or 72 hours with 4-MU and 4-MUG examined using flow cytometry.

FIG. 2D shows fluorescence of 4-MU and 4-MUG signal from 4-MU and 4-MUG treated B16F10 cells pre- and post-permeabilization examined using flow cytometry.

FIG. 3 shows the results of mice treated with 4-MU and 4-MU signal on different cell subsets in the blood analyzed by flow cytometry, as measured in the Pacific Blue channel, before and 2, 7, and 14 days after start of treatment. Bold histograms depict signal in 4-MU treated mice, shaded histograms depict background Pacific Blue signal in untreated mice.

FIG. 4A shows 4-MU and 4-MUG concentrations in serum of untreated control mice and 4-MU and 4-MUG treated mice using LC-MS/MS.

FIG. 4B shows the calculated molar ratio of 4-MU and 4-MUG in serum of untreated control mice and 4-MU and 4-MUG treated mice.

FIG. 4C shows 4-MU and 4-MUG concentrations in pancreas of untreated control mice and 4-MU and 4-MUG treated mice using LC-MS/MS.

FIG. 4D shows the calculated molar ratio of 4-MU and 4-MUG in pancreas of untreated control mice and 4-MU and 4-MUG treated mice.

FIG. 4E shows 4-MU and 4-MUG concentrations in fat of untreated control mice and 4-MU and 4-MUG treated mice using LC-MS/MS.

FIG. 4F shows the calculated molar ratio of 4-MU and 4-MUG in fat of untreated control mice and 4-MU and 4-MUG treated mice.

FIG. 4G shows 4-MU and 4-MUG concentrations in liver of untreated control mice and 4-MU and 4-MUG treated mice using LC-MS/MS.

FIG. 4H shows the calculated molar ratio of 4-MU and 4-MUG in liver of untreated control mice and 4-MU and 4-MUG treated mice.

FIG. 4I shows 4-MU and 4-MUG concentrations in muscle of untreated control mice and 4-MU and 4-MUG treated mice using LC-MS/MS.

FIG. 4J shows the calculated molar ratio of 4-MU and 4-MUG in muscle of untreated control mice and 4-MU and 4-MUG treated mice.

FIG. 5A illustrates the structures of 4-MU, 4-MUG, and a non-hydrolyzable version of 4-MUG.

FIG. 5B shows HA production by B16F10 cells cultured for 48 hours in 4-MU, 4-MUG or non-hydrolyzable 4-MUG.

FIG. 5C shows HA production by CHO-HAS3 cells engineered to over-express HA in conjunction with HAS3 synthesis cultured for 48 hours in 4-MU, 4-MUG or non-hydrolyzable 4-MUG.

FIG. 6A shows representative HA staining of pancreatic tissue from untreated DORmO mice (control), DORmO mice fed 4-MU and DORmO mice fed 4-MUG, at 12 weeks of age.

FIG. 6B shows blood glucose of untreated DORmO mice, and DORmO mice fed 4-MU and 4-MUG, beginning at 5 weeks of age, and maintained on 4-MU and 4-MUG for 15 weeks.

FIG. 6C shows representative FoxP3 staining of pancreatic islet tissue from untreated (control) and 4-MU treated DORmO mice.

FIG. 6D shows CD3+ cells in splenocytes isolated from mice that were treated with 4-MU (0.5 mg i.p.) or 4-MUG (1 mg i.p.) daily for 14 days, as analyzed by flow cytometry.

FIG. 6E shows CD4+ amongst CD3+ cells in splenocytes isolated from mice that were treated with 4-MU (0.5 mg i.p.) or 4-MUG (1 mg i.p.) daily for 14 days, as analyzed by flow cytometry.

FIG. 6F shows Foxp3+ amongst CD3+/CD4+ cells in splenocytes isolated from mice that were treated with 4-MU (0.5 mg i.p.) or 4-MUG (1 mg i.p.) daily for 14 days, as analyzed by flow cytometry.

FIG. 6G shows Foxp3+ MFI amongst CD3+/CD4+ cells in splenocytes isolated from mice that were treated with 4-MU (0.5 mg i.p.) or 4-MUG (1 mg i.p.) daily for 14 days, as analyzed by flow cytometry.

FIG. 7A shows representative images, blood glucose (BG) values, and weights (Wt) for 15 week-old db/db mice on either control chow or 4-MU chow for 10 weeks as well as for a db/+ littermate, provided for comparison.

FIG. 7B shows random (fed) BG values for 15 week old db/db mice fed either control chow, 4-MU chow, or 4-MUG in drinking water for 10 weeks as well as db/+ littermate controls fed control chow.

FIG. 7C shows weights for the mice in FIG. 7B, where each dot represents 1 mouse.

FIG. 7D shows BG levels for db/db mice maintained on control chow, 4-MU chow, or 4-MUG in drinking water starting at 5 weeks of age.

FIG. 7E shows weights for db/db mice maintained on control chow, 4-MU chow, or 4-MUG in drinking water starting at 5 weeks of age.

FIG. 7F shows intra-peritoneal glucose tolerance testing (IPGTT) of fasting db/db mice fed 4-MU for 2 weeks.

FIG. 7G shows intra-peritoneal glucose tolerance testing (IPGTT) of fasting db/db mice fed 4-MUG for 2 weeks.

FIG. 7H shows HA staining in pancreatic islets in B6 mice.

FIG. 7I shows HA staining in pancreatic islets in db/db control mice

FIG. 7J shows HA staining in pancreatic islets in db/db mice fed 4-MU.

FIG. 7K shows inhibition of HA synthesis by a beta cell line observed in vitro.

FIG. 8A is a table of 4-MUG's chemical stability assessment.

FIG. 8B is a graph that depicts 4-MUG's chemical stability as area ratio versus time in minutes.

FIG. 8C is a graph that depicts 4-MUG's chemical stability in percent remaining versus time in minutes.

### DETAILED DESCRIPTION

The present disclosure describes a critical role for the extracellular matrix molecule HA in proliferative, autoimmune, and inflammatory diseases and disorders, and the identification of a compound that inhibits HA synthesis, in particular 4-MUG. The disclosure describes the use of 4-MUG as a novel therapeutic to abrogate autoimmunity and the use of 4-MUG for treating an autoimmune, inflammatory, fibrotic, or proliferative disease or disorder, for example, cancer, type 1 diabetes, type 2 diabetes, and stroke.

Unless specifically defined herein, all terms used herein have the same meaning as they would to one skilled in the art of the present disclosure. The following definitions are provided in order to provide clarity with respect to the terms as they are used in the specification and claims to describe the claimed subject matter.

As used herein, the term “regulatory T-cells” or “Treg” cells refers to T-cells which express the cell surface markers CD4+ and CD25+, which express FoxP3 protein as measured by a Western blot and/or FoxP3 mRNA transcript.

As used herein, the term “antigen-specific regulatory T-cells” or “antigen-specific Tregs” refers to Treg cells that were induced in the presence of an antigen and which express the cell surface markers CD4+ and CD25+, which express FoxP3 protein as measured by a Western blot and/or FoxP3 mRNA transcript.

The subject can be a human or non-human animal, a vertebrate, and is typically an animal, including but not limited to, cows, pigs, horses, chickens, cats, dogs, and the like. More typically, the subject is a mammal, and in a particular embodiment, human.

As used here, a “proliferative disease” is a tumor disease, or cancer, and/or any metastases, wherever the tumor or the metastasis are located, more especially a tumor selected from the group comprising melanoma, insulinoma, lymphoma, and ovarian cancer, from cancers of the breast, colon, liver, thyroid, lung, stomach, esophagus, gall bladder, kidney, uterus, bladder, thyroid, brain, or bone and, in a broader sense, cancer types where hyaluronan has been noted to be increased.

As used herein, an “autoimmune disease” is a disease or disorder arising from and directed against an individual's own tissues. Examples of autoimmune diseases or disorders include, but are not limited to, multiple sclerosis, arthritis (rheumatoid arthritis, juvenile rheumatoid arthritis, psoriatic arthritis), conditions involving infiltration of T-cells and chronic inflammatory responses, autoimmune myocarditis, pemphigus, type 1 diabetes (also referred to as autoimmune diabetes or insulin-dependent diabetes mellitus (IDDM)), autoimmune lung disease, and the like.

As used herein, an “inflammatory disease” is a disease or disorder arising from an inflammatory state including, but not limited to, diabetes (such as type 2 diabetes, type 1 diabetes, diabetes insipidus, diabetes mellitus, maturity-onset diabetes, juvenile diabetes, insulin-dependent diabetes, non-insulin dependent diabetes, malnutrition-related diabetes, ketosis-prone diabetes or ketosis-resistant diabetes); stroke; nephropathy (such as glomerulonephritis or acute/chronic kidney failure); obesity (such as hereditary obesity, dietary obesity, hormone related obesity or obesity related to the administration of medication); hearing loss (such as that from otitis externa or acute otitis media); fibrosis related diseases (such as pulmonary interstitial fibrosis, renal fibrosis, cystic fibrosis, liver fibrosis, wound-healing or burn-healing, wherein the burn is a first-, second- or third-degree burn and/or a thermal, chemical or electrical burn); arthritis (such as rheumatoid arthritis, rheumatoid spondylitis, osteoarthritis or gout); an allergy; allergic rhinitis; acute respiratory distress syndrome; asthma; bronchitis; an inflammatory bowel disease (such as irritable bowel syndrome, mucous colitis, ulcerative colitis, Crohn's disease, gastritis, esophagitis, pancreatitis or peritonitis); or an autoimmune disease (such as scleroderma, systemic lupus erythematosus, Sjogren's syndrome, Hashimoto's thyroiditis, myasthenia gravis, transplant rejection, endotoxin shock, sepsis, psoriasis, eczema, dermatitis, or multiple sclerosis).

As used herein the term “treating” or “treatment” means the administration of a compound according to the disclosure to effectively prevent, repress, or eliminate at least one symptom associated with an autoimmune, inflammatory, fibrotic, or proliferative disease or disorder. Preventing at least one symptom involves administering a treatment to a subject prior to onset of the symptoms associated with clinical disease. Repressing at least one symptom involves administering a treatment to a subject after clinical appearance of the disease.

As used herein, the expression “effective amount” or “therapeutically effective amount” refers to an amount of the compound of the present disclosure that is effective to achieve a desired therapeutic result, such as, for example, the prevention, amelioration, or prophylaxis of a proliferative, autoimmune or inflammatory disease or disorder. The compound of the present disclosure can be administered as a pharmaceutical composition comprising a therapeutically effective amount of the compound together with a pharmaceutically acceptable carrier. In the context of the present disclosure, a “therapeutically effective amount” is understood as the amount of a compound inhibiting the synthesis, expression, and/or activity of an identified HA polymer that is necessary to achieve the desired effect which, in this specific case, is treating an autoimmune disease or disorder, in particular, multiple sclerosis. Generally, the therapeutically effective amount of the compound according to the present disclosure to be administered will depend, among other factors, on the individual to be treated, on the severity of the disease the individual suffers, on the chosen dosage form, and the like. For this reason, the doses mentioned in the present disclosure must be considered only as a guideline for a person skilled in the art, and the skilled person must adjust the doses according to the previously mentioned variables.

Therapeutically effective amounts of the compounds will generally range up to the maximally tolerated dosage, but the concentrations are not critical and can vary widely. The precise amounts employed by the attending physician will vary, of course, depending on the compound, route of administration, physical condition of the patient and other factors. The daily dosage can be administered as a single dosage or can be divided into multiple doses for administration.

The amount of the compound actually administered will be a therapeutically effective amount, which term is used herein to denote the amount needed to produce a substantial beneficial effect. Effective doses can be extrapolated from dose-response curves derived from in vitro or animal model test systems. The animal model is also typically used to determine a desirable dosage range and route of administration. Such information can then be used to determine useful doses and routes for administration in humans or other mammals. The determination of an effective dose is well within the capability of those skilled in the art. Thus, the amount actually administered will be dependent upon the individual to which treatment is to be applied, and will preferably be an optimized amount such that the desired effect is achieved without significant side-effects.

Therapeutic efficacy and possible toxicity of the compounds of the disclosure can be determined by standard pharmaceutical procedures, in cell cultures or experimental animals (e.g., ED50, the dose therapeutically effective in 50% of the population; and LD50, the dose lethal to 50% of the population). The dose ratio between therapeutic and toxic effects is the therapeutic index, and it can be expressed as the ratio LD50 to ED50. Modified therapeutic drug compounds that exhibit large therapeutic indices are particularly suitable in the practice of the methods of the disclosure. The data obtained from cell culture assays and animal studies can be used in formulating a range of dosage for use in humans or other mammals. The dosage of such compounds lies preferably within a range of circulating concentrations that include the ED50 with little or no toxicity. The dosage typically varies within this range depending upon the dosage form employed, sensitivity of the patient, and the route of administration. Thus, optimal amounts will vary with the method of administration, and will generally be in accordance with the amounts of conventional medicaments administered in the same or a similar form. Nonetheless, a compound according to the present disclosure can be administered one or more times a day, for example, 1, 2, 3, or 4 times a day, in a typical total daily amount comprised between 0.1 μg to 10,000 mg/day, typically 100 to 1,500 mg/day.

The compounds of the disclosure can be administered alone, or in combination with one or more additional therapeutic agents. Appropriate amounts in each case will vary with the particular agent, and will be either readily known to those skilled in the art or readily determinable by routine experimentation.

Administration of the compounds of the disclosure is accomplished by any effective route, for example, parenteral, topical, or oral routes. Methods of administration include inhalational, buccal, intramedullary, intravenous, intranasal, intrarectal, intraocular, intraabdominal, intraarterial, intraarticular, intracapsular, intracervical, intracranial, intraductal, intradural, intralesional, intramuscular, intralumbar, intramural, intraocular, intraoperative, intraparietal, intraperitoneal, intrapleural, intrapulmonary, intraspinal, intrathoracic, intratracheal, intratympanic, intrauterine, intravascular, and intraventricular administration, and other conventional means. The compounds of the disclosure having anti-tumor activity can be injected directly into a tumor, into the vicinity of a tumor, into a blood vessel that supplies blood to the tumor, or into lymph nodes or lymph ducts draining into or out of a tumor.

The emulsion, microemulsion, and micelle formulations of the disclosure can be nebulized using suitable aerosol propellants that are known in the art for pulmonary delivery of the compounds.

The compounds of the disclosure can be formulated into a composition that additionally comprises suitable pharmaceutically acceptable carriers, including excipients and other compounds that facilitate administration of the compound to a subject. Further details on techniques for formulation and administration can be found in the latest edition of “Remington's Pharmaceutical Sciences” (Maack Publishing Co., Easton, Pa.).

Compositions for oral administration can be formulated using pharmaceutically acceptable carriers well known in the art, in dosages suitable for oral administration. Such carriers enable the compositions containing the compounds of the disclosure to be formulated as tablets, pills, dragees, capsules, liquids, gels, syrups, slurries, suspensions, suitable for ingestion by a subject. Compositions for oral use can be formulated, for example, in combination with a solid excipient, optionally grinding the resulting mixture, and processing the mixture of granules, after adding suitable additional compounds, if desired, to obtain tablets or dragee cores. Suitable excipients include carbohydrate or protein fillers. These include, but are not limited to, sugars, including lactose, sucrose, mannitol, or sorbitol, starch from corn, wheat, rice, potato, or other plants; cellulose such as methyl cellulose, hydroxypropylmethyl-cellulose, or sodium carboxymethylcellulose; and gums including arabic and tragacanth; as well as proteins, such as gelatin and collagen. If desired, disintegrating or solubilizing agents can be added, such as the crosslinked polyvinyl pyrrolidone, agar, alginic acid, or a salt thereof, such as sodium alginate.

Dragee cores are provided with suitable coatings such as concentrated sugar solutions, which can also contain gum arabic, talc, polyvinylpyrrolidone, carbopol gel, polyethylene glycol, and/or titanium dioxide, lacquer solutions, and suitable organic solvents or solvent mixtures. Dyestuffs or pigments can be added to the tablets or dragee coatings for product identification or to characterize the quantity of active compound (i.e., dosage).

Compounds for oral administration can be formulated, for example, as push-fit capsules made of gelatin, as well as soft, sealed capsules made of gelatin and a coating such as glycerol or sorbitol. Push-fit capsules can contain the compounds mixed with filler or binders such as lactose or starches, lubricants such as talc or magnesium stearate, and, optionally, stabilizers. In soft capsules, the covalent conjugates can be dissolved or suspended in suitable liquids, such as fatty oils, liquid paraffin, or liquid polyethylene glycol with or without stabilizers.

For topical or nasal administration, penetrants appropriate to the particular barrier to be permeated are typically used in the formulation. Examples of these are 2-pyrrolidone, N-methyl-2-pyrrolidone, dimethylacetamide, dimethyl-formamide, propylene glycol, methyl or isopropyl alcohol, dimethyl sulfoxide, and azone. Additional agents can further be included to make the formulation cosmetically acceptable. Examples of these are fats, waxes, oils, dyes, fragrances, preservatives, stabilizers, and surface-active agents. Keratolytic agents such as those known in the art can also be included. Examples are salicylic acid and sulfur. For topical administration, the composition can be in the form of a transdermal ointment or patch for systemic delivery of the compound and can be prepared in a conventional manner (see, e.g., Barry, Dermatological Formulations (Drugs and the Pharmaceutical Sciences—Dekker); Harry's Cosmeticology (Leonard Hill Books).

For rectal administration, the compositions can be administered in the form of suppositories or retention enemas. Such compositions can be prepared by mixing the compounds with a suitable non-irritating excipient that is solid at ordinary temperatures but liquid at the rectal temperature and will therefore melt in the rectum to release the drug. Suitable excipients include, but are not limited to, cocoa butter and polyethylene glycols.

The amounts of each of these various types of additives will be readily apparent to those skilled in the art, optimal amounts being the same as in other, known formulations designed for the same type of administration.

Compositions containing the compounds of the disclosure can be manufactured in a manner similar to that known in the art (e.g., by means of conventional mixing, dissolving, granulating, dragee-making, levigating, emulsifying, encapsulating, entrapping or lyophilizing processes). The compositions can also be modified to provide appropriate release characteristics, sustained release, or targeted release, by conventional means (e.g., coating). As noted above, in one embodiment, the compounds are formulated as an emulsion.

Compositions containing the compounds can be provided as a salt and can be formed with many acids, including but not limited to hydrochloric, sulfuric, acetic, lactic, tartaric, malic, and succinic. Salts tend to be more soluble in aqueous or other protonic solvents than are the corresponding free base forms.

4-MU is an approved drug that has been repurposed as an inhibitor of HA synthesis including in human clinical trials, but rapid and efficient glucuronidation is thought to limit its systemic utility. As described in more detail below, the major metabolite of 4-MU, 4-MUG, actively contributes to HA synthesis inhibition in two ways. First, 4-MUG is hydrolyzed into 4-MU in serum, thereby greatly increasing the effective bioavailability of the drug. Mice fed either 4-MU or 4-MUG have equivalent ratios of 4-MU and 4-MUG in serum, liver and pancreas, indicating that there is an equilibrium in tissues between 4-MU and 4-MUG. Second, it is further shown that a non-hydrolyzable version of 4-MUG also inhibits HA synthesis, indicating that 4-MUG has direct bioactivity of its own independent of its conversion to 4-MU. Consistent with these findings, oral administration of 4-MUG to mice inhibits HA synthesis, promotes FoxP3+ regulatory T-cell expansion, and prevents autoimmune diabetes in vivo. The present disclosure shows that 4-MUG contributes to the bioavailability of 4-MU and that effective tissue drug levels of 4-MU at steady state are substantially higher than previously suspected.

There is great interest in inhibition of HA synthesis given the critical role of HA in cancer, fibrosis, diabetes, inflammation, and other settings. For example, there are data linking HA, which is the target of 4-MUG, in stroke (Tang, S. C., J. Neuroinflammation 2014; 11:101; Krupinski J, J. Biomark Insights 2008; 12(2):361-367).

To date, the only molecule known to inhibit HA synthesis is 4-MU. As described in more detail below, the inventors have discovered that 4-methylumbelliferone-glucoronide (4-MUG), a metabolite of 4-MU, can directly inhibit HA synthesis. This activity is not simply the result of conversion into 4-MU, as demonstrated using a non-hydrolyzable version of 4-MUG. Moreover, data show that 4-MUG inhibits HA synthesis at a fraction of the concentration of 4-MU. These findings are unexpected given that there are very few glucoronides that have biological activity independent of their parent molecules. Further, given the great interest in inhibition of HA for therapeutic and experimental applications, these findings have substantial potential utility in a variety of indications where HA contributes to disease pathogenesis, including diabetes, cancer, stroke, inflammation, fibrosis, and autoimmunity.

4-MUG is an Active Metabolite of 4-MU and Inhibits HA Synthesis

4-MUG is an active metabolite of 4-MU and inhibits HA synthesis. 4-MUG is a major metabolite of 4-MU. Unpublished data indicate that 4-MUG and newly described derivatives of 4-MUG are actually pharmacologically active. Indeed, 4-MUG inhibits HA synthesis by human cell lines just as well as the parent drug, 4-MU. Some derivatives of 4-MUG are likewise pharmacologically active. Not all derivatives of 4-MUG are active against HA synthesis. This is an exciting and previously unknown finding that suggests it may be possible to deliver 4-MUG or 4-MUG derivatives as an agent to inhibit HA synthesis.

It is unclear how 4-MUG inhibits HA synthesis. Typically, glucoronidation is a metabolic step that promotes the excretion and clearance of most drugs. Indeed, because glucoronidated compounds are often more water soluble, they typically do not enter cells as well as more lipophilic parent compounds. Thus it is not obvious nor intuitive that 4-MUG or its derivatives would inhibit HA synthesis.

4-MUG Inhibits HA Synthesis In Vitro

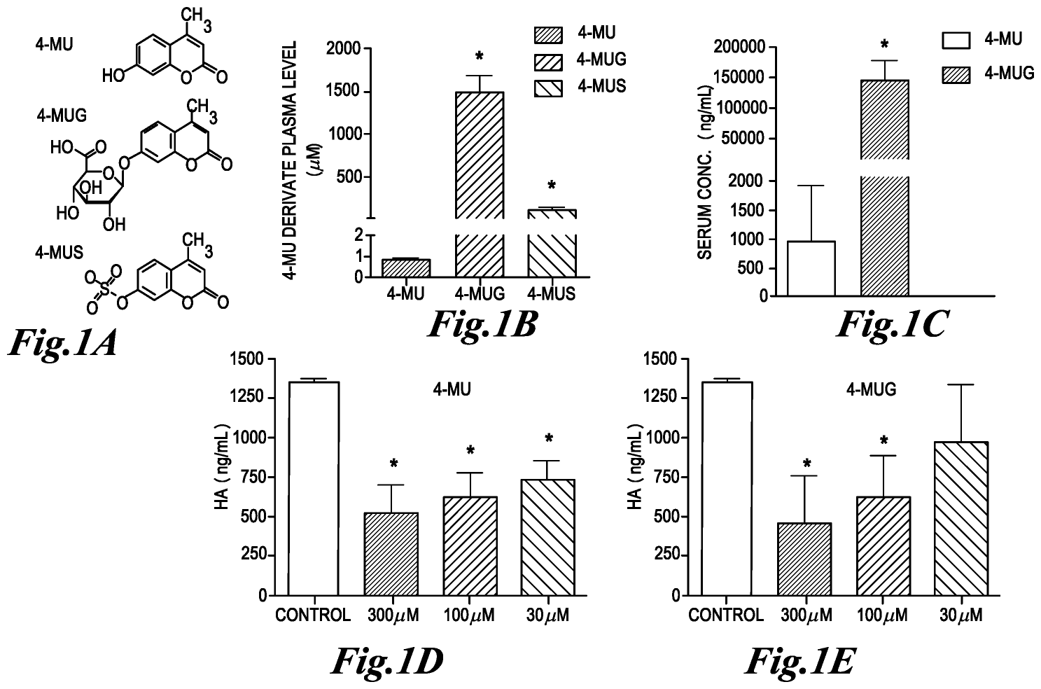

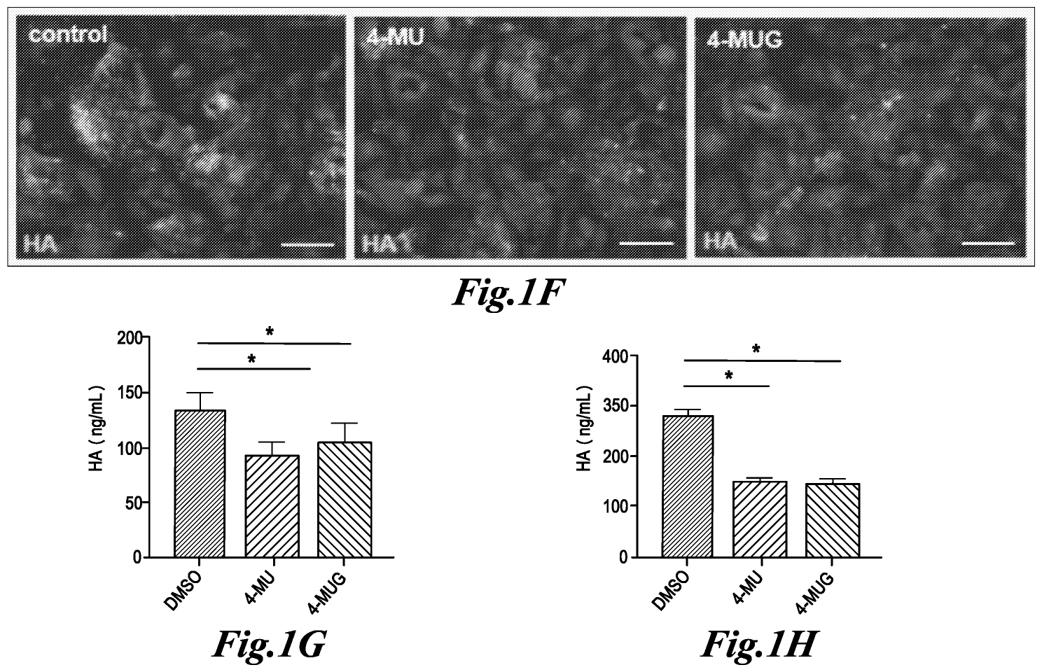

As a result of efficient glucuronidation of 4-MU in the liver and intestines by multiple UDP-glucuronosyltransferases (UGTs), the predominant form present systemically in mice on oral 4-MU chow is 4-MUG, as has been reported previously (Mulder G J, et al., Biochem Pharmacol. 1985; 34(8):1325-1329). For example, FIG. 1A illustrates molecular structures for 4-MU and its primary metabolites, 4-MUG and 4-MUS. FIG. 1B shows concentrations of 4-MU and its metabolites in plasma of animals fed 4-MU chow for two weeks, measured via HPLC. N=3 animals per group. FIG. 1C shows different concentrations of 4-MU and 4-MUG in the serum of mice fed 4-MU for two weeks measured via HPLC. N=3 animals per group. As shown in FIG. 1C, the median serum concentration of 4-MUG was about 150-fold higher than the parent compound 4-MU.

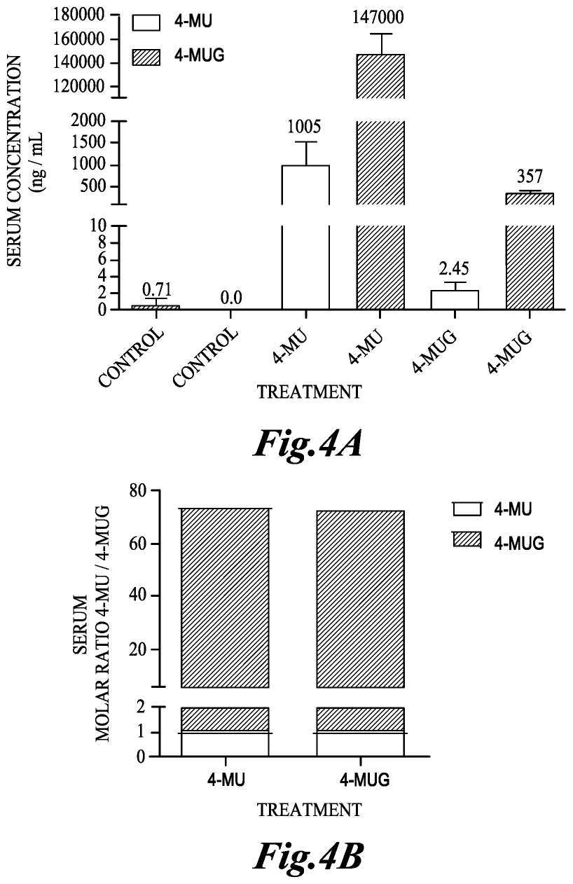

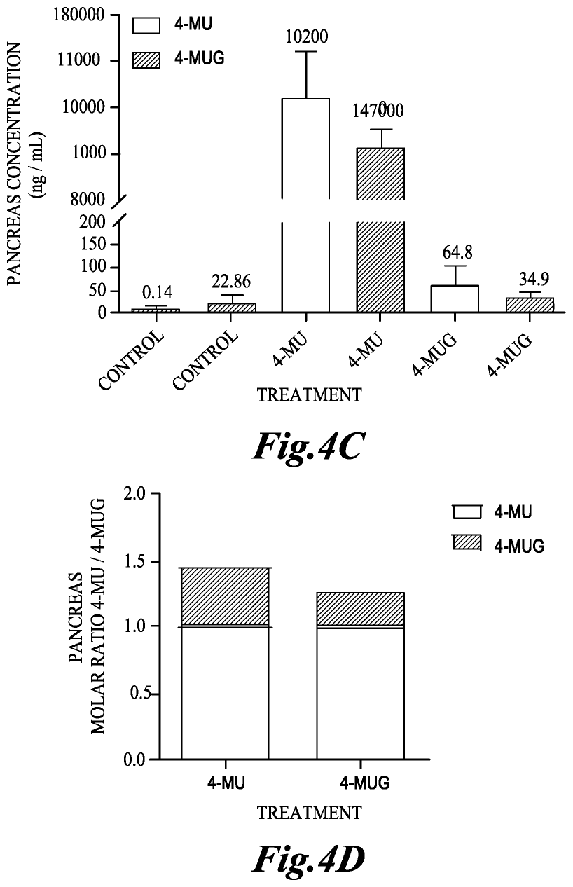

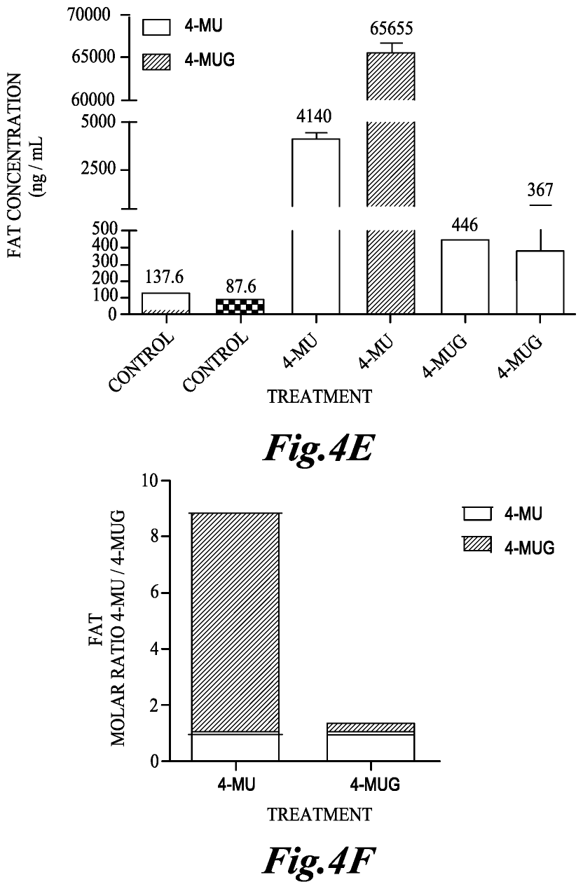

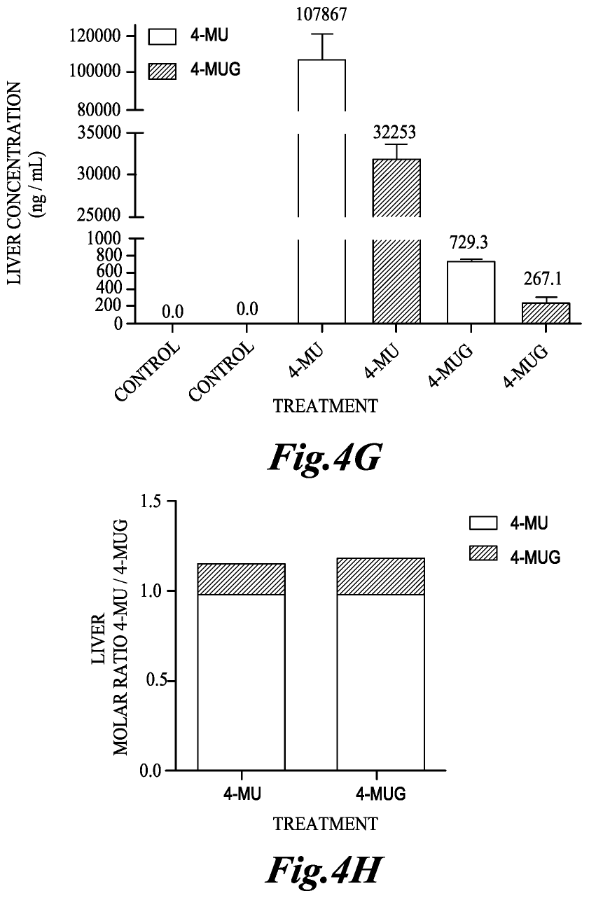

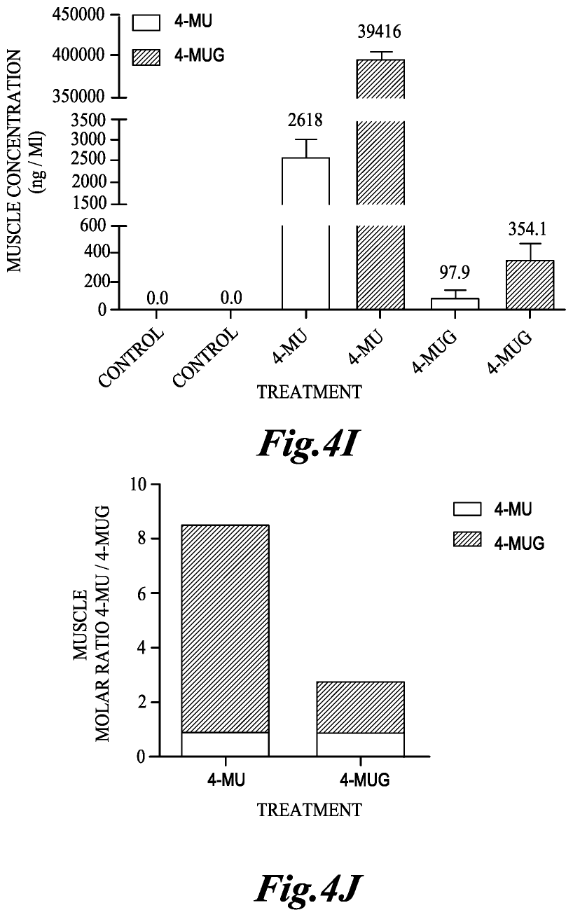

Because the activity of metabolites is an important variable in pharmacodynamic determinations, the role of the main 4-MU metabolite 4-MUG in HA synthesis inhibition was investigated. To test this, murine melanoma cells (B16F10), a cell line known to produce abundant HA, were used. A concentration dependent inhibition of HA synthesis in both 4-MU and 4-MUG treated B16F10 cells after 48 hours of drug exposure was observed. FIGS. 4D and 4E show HA production by B16F10 cells cultured for 48 hours in 4-MU (FIG. 1D) or 4-MUG (FIG. 1E). FIG. 1F shows representative images of HA staining in B16F10 cells cultured in DMSO as control (left), 4-MU (middle) or 4-MUG (right). Data represent mean±SEM; *, p<0.05 by unpaired t test. Similar findings were seen as well in primary lymphocytes. Fluorescent staining of these cells using HA binding protein (HABP), indicated that treatment with 4-MU and 4-MUG both reduced HA, as shown in FIG. 1F. Together these results demonstrate that treatment with either 4-MU or 4-MUG inhibits HA synthesis.

4-MUG is Hydrolyzed into 4-MU within Cells

Given the established activity of 4-MU leading to inhibition of HA synthesis, it seemed possible that the bioactivity of 4-MUG could be attributed to its hydrolysis into 4-MU. It is generally known that 4-MU is fluorescent while 4-MUG is not. In particular, 4-MU has an excitation wavelength of 380 nm and an emission wavelength of 454 nm in water.

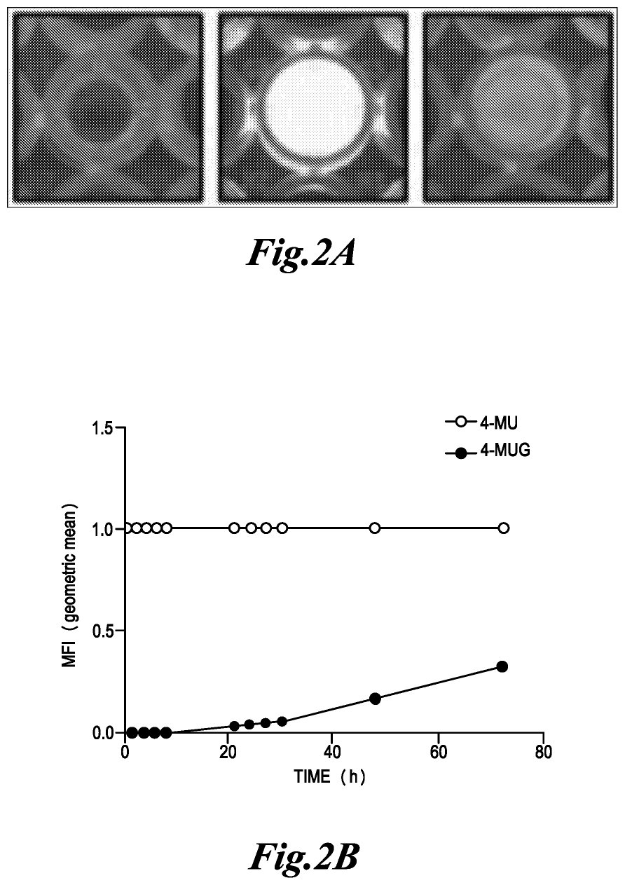

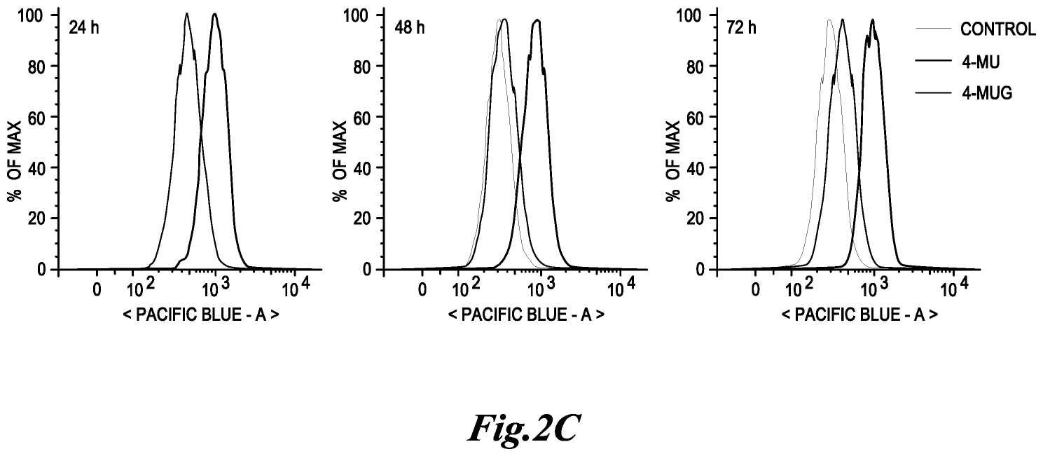

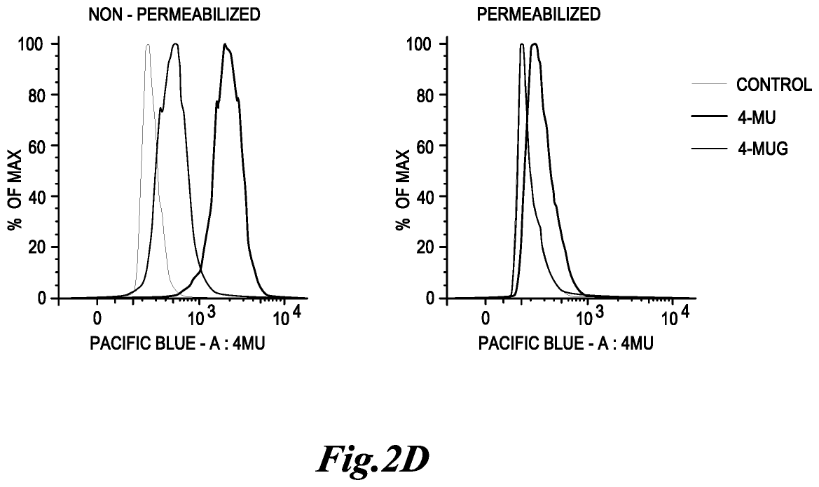

FIG. 2A shows fluorescence visualization in wells of a 96-well plate which was filled with 200 μl PBS and 10% FCS, in some wells 4-MU (middle) and 4-MUG (right) were added, control wells remained untreated (left). FIG. 2B shows fluorescent signal over time measured as mean fluorescent intensity (MFI) after 4-MU and 4-MUG were separately added to DMEM. Fluorescent values of 4-MUG were normalized to the 4-MU fluorescence. Referring to FIG. 2 B, 4-MU or 4-MUG was added to PBS with 10% FCS and the increase of fluorescence signal using a fluorescence plate reader was monitored at intervals up to 72 hours. As expected, 4-MU had a fluorescent signal at baseline. Fluorescence of 4-MUG on the other hand could only be detected starting around 30 hours, as shown in FIG. 2B. FIG. 2C shows fluorescence of 4-MU and 4-MUG from B16F10 cells incubated for 24, 48 or 72 hours with 4-MU and 4-MUG examined using flow cytometry. Referring to FIG. 2C, 4-MU and 4-MUG were added to B16F10 cells, and it was found that cells treated with 4-MUG became fluorescent after 48-72 hours, as shown in FIG. 2C. FIG. 2D shows fluorescence of 4-MU and 4-MUG signal from 4-MU and 4-MUG treated B16F10 cells pre- and post-permeabilization examined using flow cytometry. As shown in FIG. 2D, the fluorescence of these cells was lost upon permeabilization, suggesting that most of the fluorescent 4-MU is inside the cell.

Together, these data show that 4-MUG is taken up by cells and converted back into 4-MU resulting in its effects on HA synthesis inhibition. However, because the conversion of 4-MUG to 4-MU also takes place in vitro in media alone, it is apparent that extracellular conversion occurs as well.

4-MU is Taken Up by Lymphocytes

In order to determine whether 4-MU and 4-MUG are taken up by circulating cells and tissues in vivo, and using the fluorescence of 4-MU as a biomarker of 4-MU uptake, the 4-MU signal on cells isolated from spleen tissue and blood of mice that had been on oral 4-MU treatment for at least 14 days was assessed. Using the Pacific Blue channel, 4-MU signal was observed on splenocytes and circulating leukocytes from mice that were treated with 4-MU, indicating that 4-MU is taken up by cells within lymphatic tissues in vivo as well as binding to the extracellular matrix (data not shown).

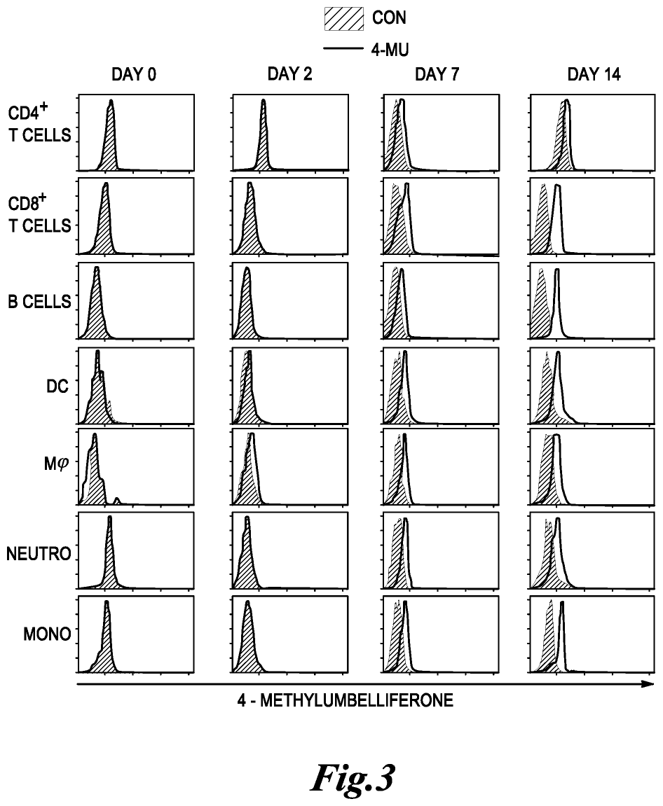

Next, the uptake of 4-MU by different leukocyte subsets was examined. To this end, mice were fed 4-MU and 4-MU signal was examined on blood leukocytes from representative animals at intervals of 0, 2, 7, and 14 days after the initiation of 4-MU treatment. FIG. 3 shows the results of mice treated with 4-MU and 4-MU signal on different cell subsets in the blood analyzed by flow cytometry, as measured in the Pacific Blue channel, before and 2, 7 and 14 days after start of treatment. Bold histograms depict signal in 4-MU treated mice, shaded histograms depict background Pacific Blue signal in untreated mice. Cell surface markers were stained to examine 4-MU uptake by multiple cell types, including CD4+ T-cells (CD3+CD4+), CD8+ T-cells (CD3+CD8+), B-cells (I-A/I-E+B220+) dendritic cells (DC; I-A/I-E+CD11c+), macrophages (Mc); I-A/I-E+CD11c−), neutrophils (Ly6G/C+CD14+) and monocytes (Ly6G/C-CD14+).

Referring again to FIG. 3, the fluorescent 4-MU signal was not seen in mice treated for 48 hours, but started to be visible after 1 week of treatment. This result is consistent with a previous report that 1-2 weeks of oral 4-MU treatment is necessary for effects on HA synthesis to become apparent (Kuipers H F, et al., Clin Exp Immunol. 2016 September; 185(3):372-81). By day 7 after 4-MU treatment, 4-MU signal is marginally visible in all of these cell populations and by day 14 all signals are decisively increased to varying extent. These data indicate that multiple leukocyte populations take up 4-MU and that a time period of between 1-2 weeks is required for this to occur. Together, these data demonstrate that 4-MU is taken up by resident cells.

In Vivo Administration of 4-MU or 4-MUG Leads to the Same Serum Ratio of 4-MU to 4-MUG

Liquid chromatography mass spectrometry (LC-MS/MS) shows that 4-MU or 4-MUG are present in tissues, and characterizes the inter-conversion between 4-MUG and 4-MU. FIGS. 4A-4J show 4-MU and 4-MUG concentrations in serum and organs from 4-MU and 4-MUG treated mice. Referring to FIG. 4, the resulting ratio between 4-MU and 4-MUG present in serum arrived at a molar ratio of 1:72. irrespective of which drug was administered (FIG. 4B), indicating the two compounds exist in equilibrium together. While the same amount of each drug was bioavailable as indicated by the same ratios, the level of 4-MU and 4-MUG were lower in the 4-MUG treated mice compared to the 4-MU treated mice (FIG. 4A) suggesting that 4-MUG was absorbed with greater efficiency. In 4-MU treated animals, higher levels of 4-MU were seen in serum than in pancreatic tissue (1005 ng/mL versus 64.8 ng/mL) (FIGS. 4A and 4C). However, substantially higher levels of 4-MU were seen in pancreatic tissue than in serum for mice fed 4-MUG (10200 ng/mL versus 2.5 ng/mL) (FIGS. 4A and 4C). The ratio of 4-MU:4-MUG in serum (1:73 for 4-MU treatment and 1:72 for 4-MUG treatment) (FIG. 4B) was far less than the ratio of 4-MU:4-MUG in pancreas (1:0.27 for 4-MU treatment and 1:0.45 for 4-MUG treatment) (FIG. 4D), suggesting that 4-MU was more efficiently bound within tissues than 4-MUG. Furthermore, 4-MU and 4-MUG concentrations were investigated in fat (FIGS. 4E and 4F), liver (FIGS. 4G and 4H), and muscle (FIGS. 4I and 4J). A similar 4-MU:4-MUG ratio was observed in fat and muscle, here the 4-MU treated animals had a significantly higher amount of 4-MUG compared to the 4-MUG treatment (FIGS. 4F and 4J). Interestingly, the liver similarly to the serum showed equilibrium between 4-MU and 4-MUG no matter what the treatment was (FIGS. 4G and 4H). The liver has a high concentration of 4-MU compared to 4-MUG independent of treatment (FIG. 4H).

Together, these data show that 4-MUG is converted into 4-MU in vivo, that 4-MU is taken up by a range of tissues and cell types in vivo, and that tissue structures serve as a reservoir for 4-MU.

A Non-Hydrolyzable Version of 4-MUG Inhibits HA Synthesis

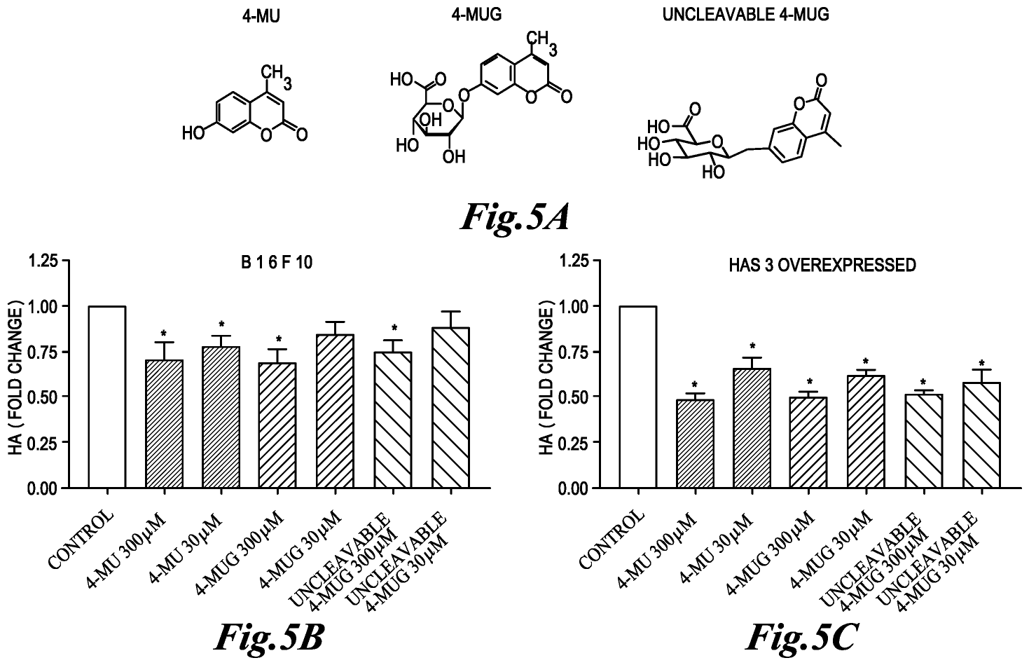

To test whether 4-MUG has bioactivity independently of its conversion to 4-MU, a non-hydrolyzable version of 4-MUG was generated. FIG. 5A illustrates the structures of 4-MU, 4-MUG, and a non-hydrolyzable version of 4-MUG. This agent, which was non-fluorescent, was not converted into fluorescent 4-MU in culture.

FIG. 5B shows HA production by B16F10 cells cultured for 48 hours in 4-MU, 4-MUG or non-hydrolyzable 4-MUG. FIG. 5C shows HA production by CHO-HAS3 cells engineered to over-express HA in conjunction with HAS3 synthesis cultured for 48 hours in 4-MU, 4-MUG or non-hydrolyzable 4-MUG. Non-hydrolyzable 4-MUG prevents HA synthesis by B16 cells (FIG. 5B), as well as by CHO cells engineered to overexpress HAS3 (FIG. 5C) at comparable doses to 4-MU or conventional 4-MUG. Together, these data demonstrate that 4-MUG inhibits HA synthesis independently of its conversion into 4-MU.

4-MUG Inhibits Diabetes Progression and Induces Foxp3 Expression in T1D Mice

To assess whether 4-MUG administration inhibited HA synthesis in vivo, as was previously shown for 4-MU (Nagy N., et al., J. Clin. Invest. 2015; 125(10):3928-3940), this drug was administered to an animal model of T1D, the D011. 10×RIPmOVA (DORmO) mouse. DORmO mice carry a T-cell receptor transgene specific for OVA (emulating autoreactive CD4+ T-cells), while simultaneously expressing OVA in conjunction with the insulin gene promoter on pancreatic beta cells (emulating the autoantigen).

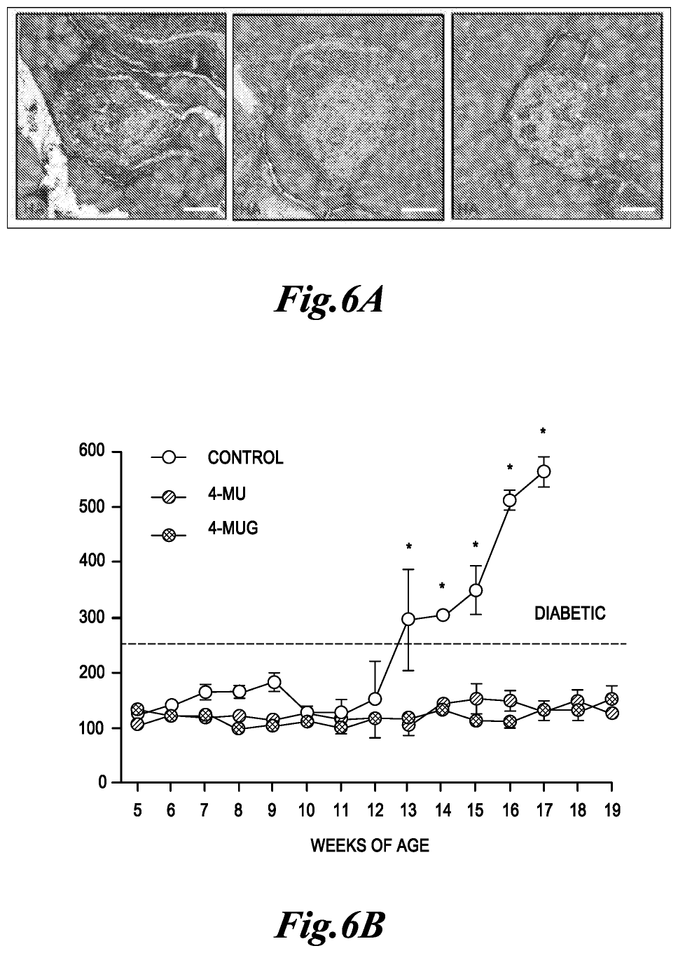

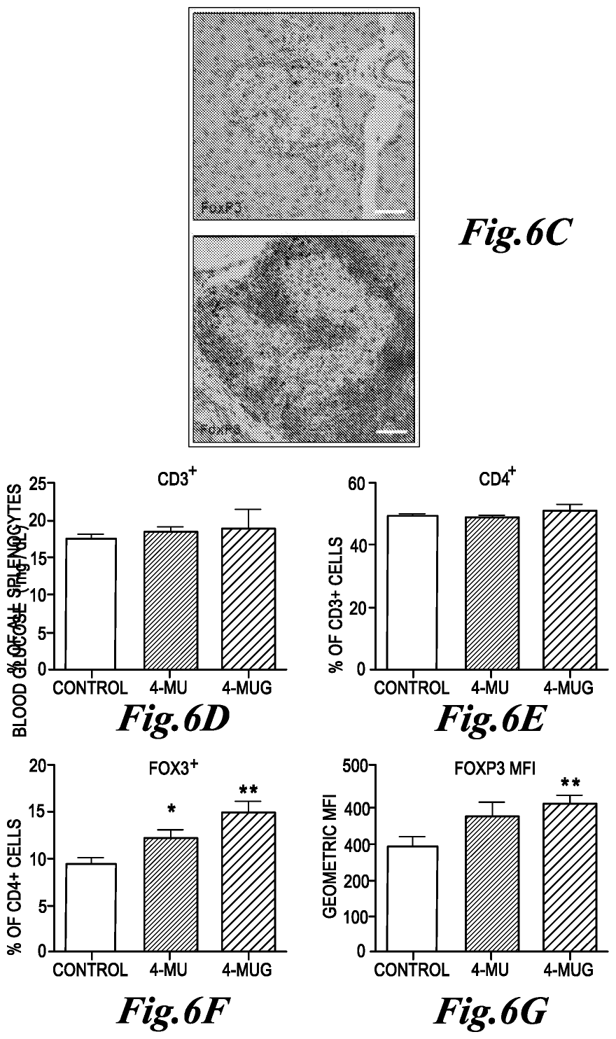

FIG. 6A shows representative HA staining of pancreatic tissue from untreated DORmO mice (control), DORmO mice fed 4-MU and DORmO mice fed 4-MUG, at 12 weeks of age. Referring to FIG. 6A, staining the DORmO islets for HA demonstrates a decrease of HA accumulation after 4-MU and 4-MUG treatment compared to untreated DORmO mice. FIG. 6B shows blood glucose of untreated DORmO mice, and DORmO mice fed 4-MU and 4-MUG, beginning at 5 weeks of age, and maintained on 4-MU and 4-MUG for 15 weeks. As shown in FIG. 6B, consistent with this, 4-MUG treatment delayed the onset of T1D as measured by blood glucose over time compared to untreated DORmO mice. In line with the normo-glycemic blood glucose, insulin positive cells were preserved in the pancreatic islets under 4-MU treatment. FIG. 6C shows representative FoxP3 staining of pancreatic islet tissue from untreated (control) and 4-MU treated DORmO mice. Original magnification, ×40. Referring to FIG. 6C, an increase of Foxp3 regulatory T-cells was observed in the pancreatic islets of the non-diabetic 4-MU treated DORmO mice. FIGS. 6D-6G show numbers of CD3+ cells, CD4+ amongst CD3+ cells and Foxp3+ amongst CD3+/CD4+ cells, in splenocytes isolated from mice that were treated with 4-MU (0.5 mg i.p.) or 4-MUG (1 mg i.p.) daily for 14 days, as analyzed by flow cytometry. *p<0.05 by unpaired t test with Welch's correction. Referring to FIGS. 6D-6G, both 4-MU and 4-MUG treatment of wild type control mice resulted in an increase of Foxp3+ regulatory T-cells, as well as an increase in their expression of Foxp3 (FIGS. 6F and 6G), CD4+ and CD3+ T-cell numbers were not affected by either treatment (FIGS. 6D and 6E). These observations are consistent with recent reports that 4-MU induces Foxp3+ Treg in multiple animal models.

As shown above, 4-MUG contributes to the bioactivity of 4-MU both in vitro and in vivo via conversion into 4-MU. Indeed, 4-MU and 4-MUG are almost equally effective over a range of concentrations at inhibiting HA synthesis by cancer cell lines in vitro. Both are likewise equally effective in treating autoimmunity in a mouse model of T1D.

Considerations of the pharmacodynamics of 4-MU must be revised to reflect the presence of 4-MUG. Animal studies have shown that 4-MUG is present at concentrations 300-fold higher than those seen for the parent molecule 4-MU. Consistent with this, in humans glucuronidation into 4-MUG accounts for over 90% of 4-MU metabolism and 93% of a single intravenous dose of 4-MU is eliminated as the 4-MUG metabolite in urine (Garrett E. R., et al., Biopharm. Drug Dispos. 1993; 14(1):13-39; Mulder G. J., et al., Biochem. Pharmacol. 1985; 34(8):1325-1329).

It is possible to administer 4-MUG to achieve the same effects as administering 4-MU both in vitro and in vivo. As described herein, in vivo experiments in the DORmO mouse model of T1D show that there is no visible difference in HA reduction in the pancreatic islets or reduction of blood glucose between 4-MU and 4-MUG treatment and both are sufficient to stop diabetes progression. This shows that 4-MUG provides an alternative therapeutic option in the treatment of autoimmune diseases. Indeed, 4-MUG has numerous advantages over 4-MU as a drug, as 4-MUG is water-soluble and can be administered, for example, in the drinking water.

It remains possible that other metabolites of 4-MU likewise are bioactive. However, these metabolites are present at such low levels (<1% of drug level (Kakizaki I., et al., J. Biol. Chem. 2004; 279(32):33281-33289)) that these are unlikely to contribute substantially to overall effects on HA.

As further described herein, tissue binding of 4-MU can be observed in vivo using 2-photon microscopy. In particular, 4-MU binds to collagen-rich structures within the tissue matrix and is also taken up by a variety of cells within the lymph nodes, pancreas, fat tissue, liver, and muscle. Accordingly, 2-photon intra-vital microscopy can be used as a novel platform for interrogating tissue binding of fluorescent drugs and that it may be possible to combine this approach with other read-outs of compound activity or tissue localization.

The fluorescent signal observed via FACS on cells is substantially diminished upon permeabilization, showing that at least some of the drug is present intra cellularly. In tissues, the fluorescent signal could be lost by treatment with collagenase or hyaluronidase, indicating that 4-MU can be bound to these molecules. These findings are corroborated by LC-MS/MS indicating that tissues indeed contain 4-MU as well 4-MUG. It is possible that the drug is incorporated into growing HA polymers but this seems unlikely, given the known mechanisms of HA synthesis. HA is normally synthesized by three HA synthases which use UDP-sugars of N-acetyl-glucosamine and glucuronic acid as precursors for HA. In the presence of 4-MU, HA synthesis is inhibited by lowering the supply of UDP glucuronic acid. 4-MU is an excellent substrate for UDP-glucuronosyltransferase (UGT), and as a result UGT consumes huge amounts of UDP-glucuronic acid, transferring the glucuronic acid onto 4-MU, thereby depleting the cellular precursor pool which leads to inhibition of HA synthesis. Therefore it is unlikely that 4-MU gets incorporated into HA during its synthesis.

Together, these studies indicate that 4-MU is more bioavailable than was previously believed due to the contributions of its metabolite 4-MUG. This insight alters the experimental and therapeutic picture for 4-MU and can facilitate the development of potential therapeutic strategies targeting HA synthesis in proliferative diseases such as cancer, autoimmunity, and other diseases and disorders. In particular, 4-MUG has therapeutic potential on its own.

### Example 1

To examine the effect of 4-MU and its metabolite 4-MUG on HA synthesis inhibition, the following experiments were performed.

Mice

All animals were bred and maintained under specific pathogen-free conditions, with free access to food and water, in the animal facilities at Stanford University Medical School (Stanford, Calif.). B6 db/db LeptR−/− mice were purchased from Jackson Laboratories (JAX) as well as DO11.10 transgenic mice. The DO11.10 mice were bred with Balb/c mice expressing RIPmOva (ovalbumin peptide amino acids 323-339; available at the Benaroya Research Institute) to generate the DORmO double-transgenic mice. In addition, C57Bl/6J mice were bred in-house at Stanford University School of Medicine.

Mouse Diabetes Monitoring

Beginning at four weeks of age, mice were weighed weekly as well as bled via the tail vein for the determination of their blood glucose level using a Contour® blood glucose meter and blood glucose monitoring strips (Bayer Healthcare). When two consecutive blood glucose readings of 250 mg/dL were recorded, animals were considered diabetic. When two consecutive blood glucose readings of 300 mg/dL were recorded, animals were euthanized.

4-MU and 4-MUG Treatment

The 4-MU (Alfa Aesar) was pressed into mouse chow (TestDiet, St. Louis, Mo.) and irradiated before shipment, as previously described (Nagy N., et al., Circulation. 2010; 122(22):2313-2322). This chow formulation delivers 250 mg/mouse/day, yielding a serum drug concentration of 640.3±17.2 nmol/L in mice, as measured by HPLC-MS. 4-MUG (ChemImpex, Wood Dale, Ill.) was distributed in the drinking water at a concentration of 2 mg/ml, delivering 10 mg/mouse/day, yielding a serum drug concentration of 357.1±72.6 ng/mL in mice, as measured by LC-MS/MS. Mice were initiated on 4-MU and 4-MUG at five, eight or twelve weeks of age, unless otherwise noted, and were maintained on this diet until they were euthanized, unless otherwise noted. For analysis of Foxp3+ regulatory T-cell numbers in naïve mice, mice were treated daily with 0.5 mg of 4-MU or 1.0 mg 4-MUG in 200 μl 0.08% carboxymethylcellulose in saline by intra-peritoneal injection.

Cell Culture

B16F10 cells were cultured in DMEM and were treated with different concentrations of 4-MU and 4-MUG (30, 100, 300 μM) for 24 and 48 hours. Cultured cells were lysed and analyzed for HA concentration determination using an HA ELISA. HA staining in B16F10 cells placed in 96 well plates were imaged using fluorescence microscopy. To measure 4-MU florescence intensity in B16F10 cells treated with 4-MU and 4-MUG, B16F10 cells were trypsinized and 4-MU fluorescence associated with the cells was analyzed by flow cytometry in the Pacific Blue channel using a BD™ LSRII flow cytometer. For permeabilization, after trypsinization, cells were incubated in methanol at −20° C. for 20 min and washed once before flow cytometric analysis.

Leukocyte 4-MU Uptake Assessments

C57Bl/6J mice were treated with 4-MU and leukocytes from representative animals were isolated from the blood at baseline (before 4-MU treatment) and at intervals of 2, 7, and 14 days after the initiation of chow. Peripheral venous blood was collected in heparin-coated tubes after cutting the tail veins of mice on 4-MU or control chow. After isolation, blood samples were centrifuged (1000×g, 4° C.) for 30 min. The serum supernatant was extracted to detect HA levels using a modified HA ELISA as previously described (Nagy N., et al., J. Clin. Invest. 2015; 125(10):3928-3940). To detect fluorescence emitted by 4-MU using flow cytometry on specific leukocyte subsets, peripheral blood red cells were lysed using Ammonium-Chloride-Potassium (ACK) buffer, and leukocytes were stained with the following fluorochrome-conjugated antibodies: BV650-CD3 (17-A2), BV785-CD4 (RM4-5), APC-CD11c (N418), PE-CD14 (Sa2-8), PE-Cy7-Ly-6G/C (RB6-8C5), PE-CyS. 5-B220 (RA3-6B2) and FITC-I-A/I-E (MHC class II) (M5/114.15.2) from BD-Biosciences (San Jose, Calif.). Cells were stained for 30 minutes at room temperature following blockage of Fc receptors (CD16/32, 2.4G2) for 10 minutes. Samples were washed once with 1 mL FACS buffer (PBS containing 2% FBS and 1 mM EDTA) and fixed with 1.6% paraformaldehyde. Samples were run on a BD™ LSRII flow cytometer (Beckon Dickinson) and data was analyzed using FlowJo software (TreeStar).

Liquid Chromatography Tandem Mass Spectrometry (LC-MS/MS) Analysis of 4-MU and 4-MUG Concentrations in Mouse Serum and Organs

4-methylumbelliferone-13C4 (Toronto Research Chemicals, Ontario, Canada) was used as the internal standard (IS) for 4-MU and 7-hydroxycoumarin β-D-glucuronide (Toronto Research Chemicals, Ontario, Canada) as the IS for 4-MUG. The neat stock solutions of 4-MU and 4-MUG were mixed and diluted in 50% methanol to prepare the spiking solutions ranging from 1 ng/mL to 5000 ng/mL for each compound.

Tissue samples were weighed and 1 volume of stainless steel bullet blender beads (Next Advance) and 3 volumes of MilliQ® water were added. Tissues were homogenized by a blender at 4° C. (Bullet blender, Next Advance) according to manufacturer's instruction. For calibration standards, 25 μl of blank serum or tissue homogenate was mixed with 25 μl of the spiking solutions. For samples to be tested, 25 μl of serum or tissue homogenate was mixed with 25 μl of 50% methanol to make up the volume. 25 μl of a mixture of the two IS (1000 ng/ml each in 50% methanol) was then added. After vortexing all standards and samples, 150 μl of methanol/acetonitrile 20:80 (v/v) was added to the mixture and the sample was further vortexed vigorously for 1 min followed by centrifugation at 3,000 rpm for 10 min. 100 μl of the supernatant was taken and diluted with 200 μl of MilliQ® water.

The LC-MS/MS system consists of an AB SCIEX QTRAP® 4000 mass spectrometer linked to a Shimadzu UFLC system. Mobile phase A is HPLC grade water. Mobile phase B is HPLC grade acetonitrile. LC separation was carried out on a Phenomenex Luna® PFP(2) column (3 μm, 150×2 mm) with isocratic elution using 45% mobile phase B and a flow rate of 0.4 ml/min at room temperature. The analysis time was 2.5 min 10 μl of the extracted sample was injected. The mass spectrometer was operated in the negative mode with the following multiple-reaction monitoring (MRM) transitions: m/z 174.7→32.9 for 4-MU, m/z 178.7-434.9 for 4-MU-13C4 (IS), m/z 350.8-474.9 for 4-MUG and m/z 336.9-460.9 for 7-hydroxy coumarin β-D-glucuronide (IS). Data acquisition and analysis were performed using the Analyst 1. 6.1 software (AB SCIEX).

Measurement of HA Levels

Samples were thawed and assayed for HA levels using a modified HA ELISA as previously described (Nagy N., et al., J. Clin. Invest. 2015; 125(10):3928-3940). Each sample was analyzed in triplicate with a mean value obtained per sample. For cell normalization, LI-COR CellTag™ 700 Stain was used according to the manufactures protocol.

Tissue Processing and Imaging

Tissues for histochemistry were extracted from the animals and immediately transferred into 10% neutral buffered formalin (NBF). The tissue was processed to paraffin on a Leica ASP300 Tissue Processor (Leica Microsystems Inc.). Then 5 μm thick sections were cut on a Leica RM 2255 Microtomes (Leica Microsystems Inc.). All staining steps were performed on a Leica Bond-Max™ automated immune histochemistry (IHC) stainer (Leica Microsystems Inc.). For HA affinity histochemistry (AFC) the Bond™ Intense R Detection kit, a streptavidin-HRP system, (Leica Microsystems, Inc.) was used with 4 μg/mL biotinylated-HABP in 0.1% BSA-PBS as the primary. The Bond™ Polymer Detection Kit was used for all other immunohistochemistry. This detection kit contains a goat anti-rabbit conjugated to polymeric HRP and a rabbit anti-mouse post primary reagent for use with mouse primaries.

For Foxp3 and insulin (anti-insulin, ab7842 abcam) sections were incubated 60 min with 8 μg/mL rat anti-Foxp3 clone FJK-16s (eBioscience). Incubation with rabbit anti rat IgG (Vector Labs), post-primary was added in lieu of the post-primary reagent from the kit.

CD3 IHC required pre-treatment using heat-mediated antigen retrieval with EDTA at high pH (Bond epitope retrieval solution 2) for 20 min. Subsequently sections were incubated with 2.5 μg/mL rabbit anti-CD3 (A0452, Dako) and detection was performed using the Bond™ Polymer Refine Detection Kit.

All images were visualized using a Leica DMIRB inverted fluorescence microscope equipped with a Pursuit 4-megapixel cooled color/monochrome charge-coupled device camera (Diagnostic Instruments). Images were acquired using the Spot™ Pursuit camera and Spot Advance Software (SPOT Imaging Solutions; Diagnostic Instruments). Image analysis was performed accordingly using Image J (NIH), as described previously (Nagy N., et al., J. Clin. Invest. 2015; 125(10):3928-40).

Mouse Splenocyte Isolation and Regulatory T-Cell Identification

Spleens were extracted from mice and cells were harvested by homogenization through a 70 μm cell strainer. Red blood cells were lysed using ACK buffer, after which the splenocyte suspensions were stained according to the protocol described above with the following fluorochrome-conjugated antibodies. V500-CD3 (500A2), BV785-CD4 (RM4-5) and A1488-Foxp3 (FJK-16s). Flow cytometry was performed on an LSRII and data analysis was done using FlowJo (Treestar).

### Example 2

4-MUG Inhibits HA Synthesis by Multiple Cancer Cells In Vitro

Because the activity of metabolites is an important variable in pharmacodynamic determinations, the effect of the main 4-MU metabolite 4-MUG on HA synthesis was investigated. To test this, melanoma cells from a cell line (B16F10) that produces abundant HA were used.

FIGS. 1D and 1E show HA production by B16F10 cells cultured for 48 hours in 4-MU (FIG. 1D) and 4-MUG (FIG. 1E). Referring to FIGS. 1D and 1E, a concentration dependent inhibition of HA synthesis was observed in both 4-MU (FIG. 1D) and 4-MUG (FIG. 1E) treated B16F10 cells after 48 hours of drug exposure. Fluorescent staining of these cells using HA binding protein (HABP), indicated that treatment with 4-MU and 4-MUG both reduced HA (FIG. 1D).

FIG. 1G shows HA synthesis inhibition upon treatment with 4-MU or 4-MUG in CTLL2 cells, and FIG. 1H shows HA synthesis inhibition upon treatment with 4 MU or 4-MUG in Min6 cells. In addition to B16F10 melanoma cells, 4-MUG likewise inhibited HA synthesis by CTLL2 cells, a lymphoma cell line, (FIG. 1G) as well as Min6 cells, an insulinoma cell line (FIG. 1H). Together these results indicate that treatment with either 4-MU or 4-MUG inhibits HA synthesis.

To test whether 4-MUG has bioactivity independently of any possible conversion to 4-MU, a non-hydrolyzable version of 4-MUG was generated (FIG. 5A). FIG. 5B shows HA production by B16F10 cells cultured for 48 hours in 4-MU, 4-MUG or non-hydrolyzable 4-MUG. FIG. 5C shows HA production by CHO-HAS3 cells engineered to over-express HA in conjunction with HAS3 synthesis cultured for 48 hours in 4-MU, 4-MUG or non-hydrolyzable 4-MUG. Non-hydrolyzable 4-MUG prevented HA synthesis by the melanoma cell line B16F10 (FIG. 5B) as well as by the ovarian cancer cell line CHO (FIG. 5C) at comparable doses to 4-MU or conventional 4-MUG.

Together, these data demonstrate that 4-MUG directly inhibits HA synthesis by four different cancer cell lines (B16F10 melanoma, Min6 insulinoma, CTLL2 lymphoma, and CHO ovarian). Moreover, these data indicate that while 4-MUG can undergo conversion to 4-MU, 4-MUG nonetheless inhibits HA synthesis directly in the absence of this conversion.

### Example 3

To assess the effect of 4-MUG administration on inhibition of HA synthesis in vivo, the drug was administered to the animal model of T1D, the DO11.10× RIPmOVA (DORmO) mouse. DORmO mice carry a T-cell receptor transgene specific for OVA (emulating autoreactive CD4+ T-cells), while simultaneously expressing OVA in conjunction with the insulin gene promoter on pancreatic beta cells (emulating the autoantigen).

FIG. 6A shows representative HA staining of pancreatic tissue from untreated DORmO mice (control), DORmO mice fed 4-MU and DORmO mice fed 4-MUG, at 12 weeks of age. Referring to FIG. 6A, staining the DORmO islets for HA shows a decrease of HA accumulation after 4-MU and 4-MUG treatment compared to untreated DORmO mice was shown. FIG. 6B shows blood glucose of untreated DORmO mice, and DORmO mice fed 4-MU and 4-MUG, beginning at 5 weeks of age, and maintained on 4-MU and 4-MUG for 15 weeks. Referring to FIG. 6B, and consistent with this, 4-MUG treatment delayed the onset of T1D as measured by blood glucose over time compared to untreated DORmO mice. FIG. 6C shows representative FoxP3 staining of pancreatic islet tissue from untreated (control) and 4-MU treated DORmO mice. Original magnification×40. Further, an increase of Foxp3 regulatory T-cells was observed in the pancreatic islets of the non-diabetic 4-MU treated DORmO mice (FIG. 6C). FIG. 6F shows Foxp3+ amongst CD3+/CD4+ cells in splenocytes isolated from mice that were treated with 4-MU (0.5 mg i.p.) or 4-MUG (1 mg i.p.) daily for 14 days, and FIG. 6G shows Foxp3+ MFI amongst CD3+/CD4+ cells in splenocytes isolated from mice that were treated with 4-MU (0.5 mg i.p.) or 4-MUG (1 mg i.p.) daily for 14 days, as analyzed by flow cytometry. Referring to FIGS. 6F and 6G, both 4-MU and 4-MUG treatment of wild type control mice produced in an increase of Foxp3+ regulatory T-cells as well as an increase in their expression of Foxp3. FIG. 6D shows CD3+ cells in splenocytes isolated from mice that were treated with 4-MU (0.5 mg i.p.) or 4-MUG (1 mg i.p.) daily for 14 days, and FIG. 6E shows CD4+ amongst CD3+ cells in splenocytes isolated from mice that were treated with 4-MU (0.5 mg i.p.) or 4-MUG (1 mg i.p.) daily for 14 days, as analyzed by flow cytometry. FIGS. 6D and 6E show that CD4+ and CD3+ T-cell numbers were not affected by either treatment. These observations are consistent with studies showing that 4-MU induces Foxp3+ Treg in multiple animal models (Nagy, N., et al., J. Clin. Invest. 125(10):3928-3940; Kuipers, H. F., et al., Proc. Natl. Acad. Sci. U.S.A. 113:1339-1344; Kuipers, H. F., et al., Clin. Exp. Immunol. 185:372-381) and indicate that 4-MUG does so as well.

### Example 4

To assess whether 4-MUG is effective against HA synthesis in a different inflammatory disease that is non-autoimmune in nature, the role of 4-MUG in the db/db model of type 2 diabetes was investigated. db/db mice lack a functional leptin receptor and are obese and diabetic.

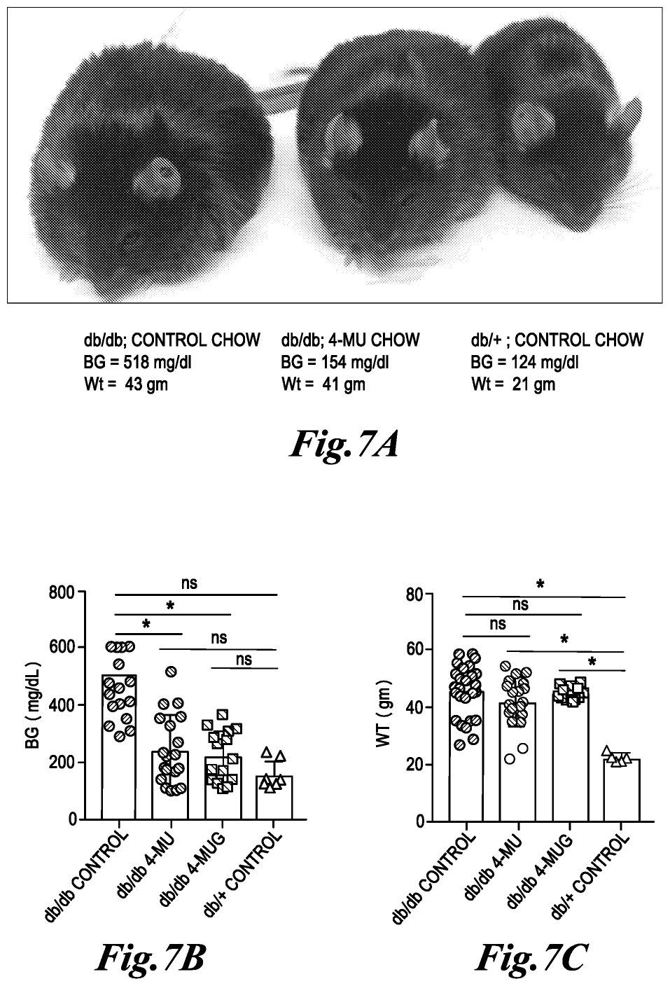

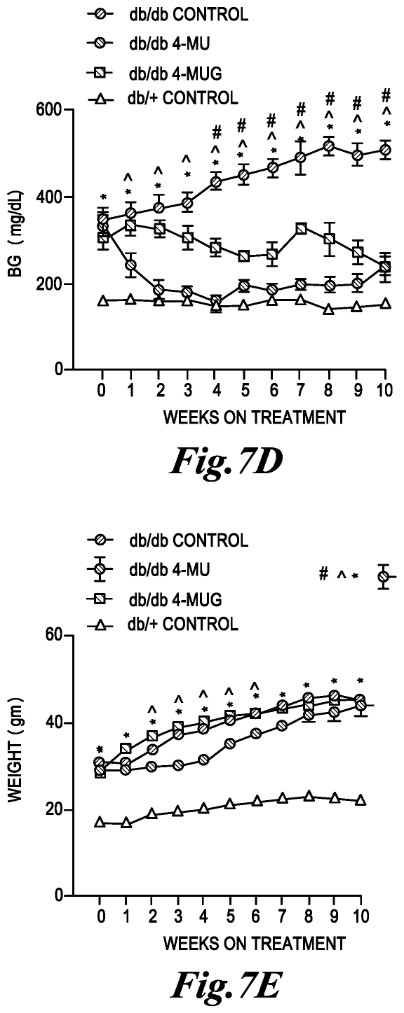

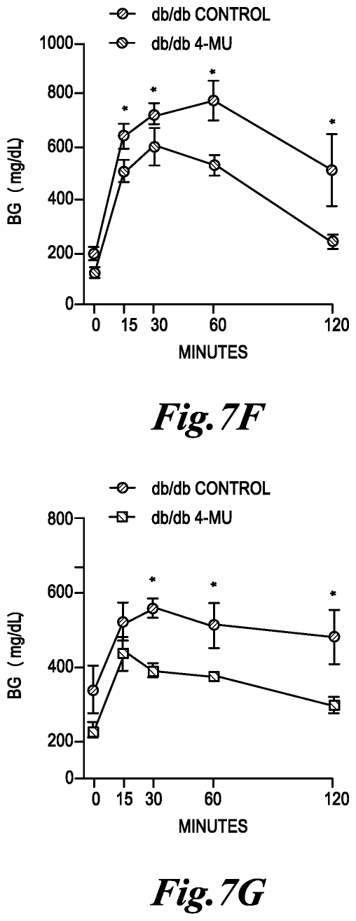

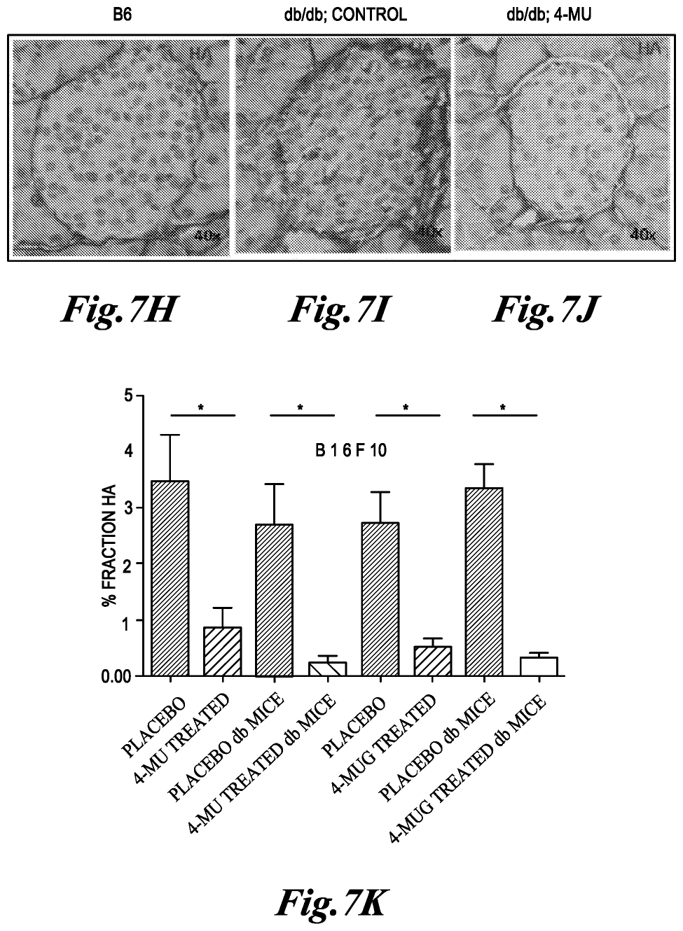

FIG. 7A shows representative images, blood glucose (BG) values, and weights (Wt) for 15-week-old db/db mice on either control chow or 4-MU chow for 10 weeks as well as for a db/+ littermate, provided for comparison. FIG. 7B shows random (fed) BG values for 15-week-old db/db mice fed either control chow, 4-MU chow, or 4-MUG in drinking water for 10 weeks as well as db/+ littermate controls fed control chow. FIG. 7C shows weights for the mice in FIG. 7B, where each dot represents 1 mouse. Referring to FIGS. 7A-7C, administration of either 4-MU or 4-MUG to db/db mice over one month reliably decreased blood glucose (BG) levels compared to age and gender (male) matched mice fed control chow. FIG. 7D shows BG levels for db/db mice maintained on control chow, 4-MU chow, or 4-MUG in drinking water starting at 5 weeks of age. Referring to FIG. 7D, the beneficial effect of 4-MU and 4-MUG on glycemic control was maintained for at least 10 weeks, indicating a lasting improvement. FIGS. 7F and 7G show intra-peritoneal glucose tolerance testing (IPGTT) of fasting db/db mice fed 4-MU or 4-MUG for 2 weeks. Referring to FIGS. 7F and 7G, this improvement in glycemic control was observed upon intra-peritoneal glucose tolerance testing (IPGTT) of fasting db/db mice fed either 4-MU or 4-MUG for the previous 2 weeks.

Without wishing to be bound by theory, one potential explanation for these data could be that db/db mice on 4-MU eat less chow and are normoglycemic as a consequence of reduced caloric intake. Indeed, db/db mice on 4-MU chow showed an initial decrease in weight for several weeks after the start of treatment. However, weights in db/db mice fed either 4-MU or 4-MUG soon recovered (FIG. 7E) whereas the observed improvements in glycemic control persisted over the same time while the separation of glucose levels with 4-MU treatment persisted throughout the phase of weight regain in these mice, and was still present long after the initiation of treatment when body weight no longer differed between groups.

Finally, these effects of 4-MU and 4-MUG on diabetes control were associated with reduced HA staining in pancreatic islets (FIGS. 7H-7K), consistent with the inhibition of HA synthesis by a beta cell line observed in vitro (FIG. 7F). Together these data indicate that both 4-MU and 4-MUG restore euglycemia in db/db mice equally well.

### Example 5

To determine the chemical stability of 4-MUG, its half-life (t_{1/2}) was evaluated. 4-MUG was tested at a concentration of 100 μM. The internal standard (IS) was prepared at 50 ng/mL with tolbutamide in DMSO and a buffer solution was prepared of fasted state simulated gastric fluid (FaSSGF) at pH 1.6. The buffer was pre-warmed at 37° C. for 15 minutes, subsequently 4-MUG was added, and the solution was vortexed. Next, 30 μL of this reaction mixture was removed at each time point for analysis. The time points included 0 minutes, 15 minutes, 30 minutes, 60 minutes and 120 minutes. The reaction was stopped at the end of the experiment by adding IS solution. The samples were centrifuged at 4,000 rpm for 15 minutes at 4° C. 100 μL of the supernatant was mixed with distilled water for further liquid chromatography with tandem mass spectrometry (LC-MS/MS) analysis. The mass spectrometry detection was performed using a SCIEX® API 4500 Qtrap® (Sciex, Redwood City, Calif.). Each compound was analyzed by reverse phase high performance liquid chromatography (HPLC). The parameters calculated were the ratio of 4-MUG, the percent of 4-MUG remaining versus time, and the estimation of 4-MUG's t_{1/2}.

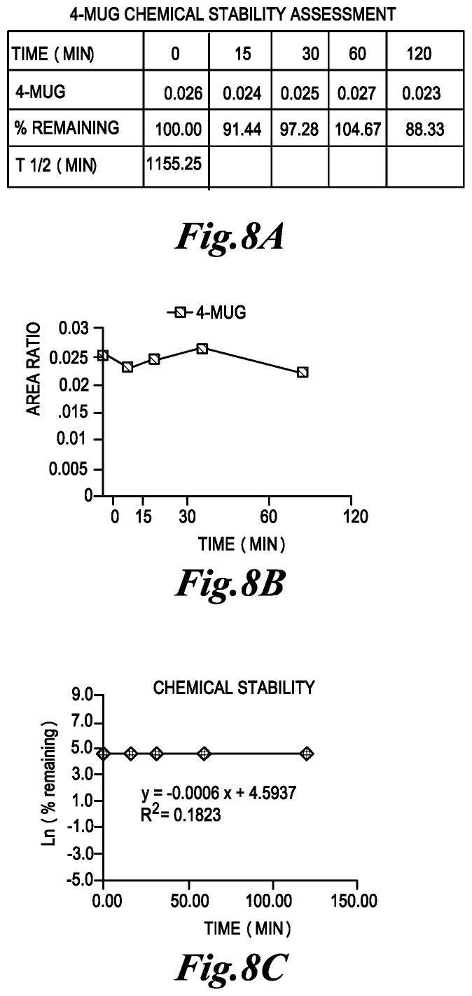

FIG. 8A is a table of 4-MUG's chemical stability assessment. FIG. 8B is a graph that depicts 4-MUG's chemical stability as area ratio versus time in minutes. FIG. 8C is a graph that depicts 4-MUG's chemical stability in percent remaining versus time in minutes. As shown in FIGS. 8A, 8B, and 8C, 4-MUG is stable under standard chemical testing for a relatively long period of time.

While illustrative embodiments have been illustrated and described, it will be appreciated that various changes can be made therein without departing from the spirit and scope of the invention.

The embodiments of the invention in which an exclusive property or privilege is claimed are defined as follows:

1. A composition for treating an autoimmune, inflammatory, fibrotic, or proliferative disease or disorder comprising (i) a compound that inhibits hyaluronan synthesis, and (ii) a pharmaceutically acceptable carrier.

2. The composition of claim 1, wherein the compound is a UDP-glycosyltransferase inhibitor.

3. The composition of claim 2, wherein the compound is a UDP-glucuronyltransferase inhibitor.

4. The composition of claim 3, wherein the compound is 4-methylumbelliferone-glucuronide.

5. The composition of claim 1, wherein the compound is effective to induce a regulatory T-cell response.

6. The composition of claim 5, wherein the compound is effective to increase FoxP3+ regulatory T-cells.

7. The composition of claim 1, wherein the autoimmune disease or disorder is selected from the group consisting of amyloidosis, ankylosing spondylitis, nephritis, antiphospholipid syndrome, autoimmune angioedema, autoimmune encephalomyelitis, autoimmune hepatitis, autoimmune orchitis, autoimmune pancreatitis, autoimmune retinopathy, autoimmune urticaria, Behcet's disease, benign mucosal pemphigoid, bullous pemphigoid, celiac disease, Chagas disease, CREST syndrome, Crohn's disease, fibromyalgia, Graves' disease, Guillain-Barre syndrome, Hashimoto's thyroiditis, hemolytic anemia, Henoch-Schonlein purpura (HSP), nephropathy, juvenile arthritis, juvenile diabetes (Type 1 diabetes), lupus, multiple sclerosis, neuromyelitis optica, polyarteritis nodosa, primary biliary cirrhosis, primary sclerosing cholangitis, psoriasis, psoriatic arthritis, rheumatoid arthritis, sarcoidosis, scleroderma, Sjogren's syndrome, temporal arteritis (giant cell arteritis), ulcerative colitis (UC), vasculitis, and vitiligo.

8. The composition of claim 1, wherein the inflammatory disease or disorder is selected from the group consisting of renal ischaemia-reperfusion injury, asthma, pulmonary hypertension, type 2 diabetes, arthritis, atherosclerosis, wound healing, chronic obstructive pulmonary disease (COPD), emphysema, bronchiolitis obliterans syndrome (BOS), allogeneic transplant rejection, graft versus host disease, dermatomyositis, inflammatory bowel disease, and stroke.

9. The composition of claim 8, wherein the inflammatory disease or disorder is type 2 diabetes, allogenic transplant rejection, or graft versus host disease.

10. The composition of claim 1, wherein the fibrotic disease or disorder is selected from the group consisting of primary sclerosing cholangitis, biliary cirrhosis, biliary spasm, cirrhosis, liver fibrosis, renal fibrosis, dermal fibrosis, intestinal fibrosis, and lung fibrosis.

11. The composition of claim 1, wherein the proliferative disease or disorder is selected from the group consisting of pancreatic cancer, prostate cancer, skin cancer, esophageal cancer, breast cancer, liver cancer, bone cancer, ovarian cancer, kidney cancer, anal cancer, brain cancer, biliary cancer, melanoma, insulinoma, endometrial cancer, stomach cancer, testes cancer, thyroid cancer, cervical cancer, and lymphoma.

12. The composition of claim 11, wherein the proliferative disease or disorder is melanoma, insulinoma, lymphoma, or ovarian cancer.

13. A method for treating an autoimmune, inflammatory, fibrotic, or proliferative disease or disorder in a mammalian subject in need thereof, the method comprising administering to the subject a composition comprising a compound in an amount effective to inhibit hyaluronan synthesis in the mammalian subject.

14. The method of claim 13, wherein the compound is a UDP-glycosyltransferase inhibitor.

15. The method of claim 14, wherein the compound is a UDP-glucuronyltransferase inhibitor.

16. The method of claim 15, wherein the compound is 4-methylumbelliferone-glucuronide.

17. The method of claim 13, wherein the compound is effective to induce a regulatory T-cell response.

18. The method of claim 17, wherein the compound is effective to increase FoxP3+ regulatory T-cells.

19. The method of claim 13, wherein the mammalian subject is a human subject.

20. A method for treating a proliferative disease and/or reversing progression of a proliferative disease in a mammalian subject suffering from or at risk of developing a proliferative disease, the method comprising:
administering to the mammalian subject a composition comprising a compound in an amount effective to inhibit hyaluronan synthesis in the mammalian subject.

21. The method of claim 20, wherein the compound is a UDP-glycosyltransferase inhibitor or a UDP-glucuronyltransferase inhibitor.

22. The method of claim 21, wherein the compound is 4-methylumbelliferone-glucuronide.

23. The method of claim 20, wherein the mammalian subject is a human subject.

24. The method of claim 20, wherein the proliferative disease is melanoma, insulinoma, lymphoma, ovarian cancer.

25. A method for treating type 1 diabetes or type 2 diabetes in a mammalian subject in need thereof, the method comprising administering to the subject a composition comprising a compound in an amount effective to inhibit hyaluronan synthesis in the mammalian subject.

26. The method of claim 25, wherein the compound is a UDP-glycosyltransferase inhibitor or a UDP-glucuronyltransferase inhibitor.

27. The method of claim 26, wherein the compound is 4-methylumbelliferone-glucuronide.

28. The method of claim 25, wherein the mammalian subject is a human subject.

29. The method of claim 25, wherein the compound is effective to induce a regulatory T-cell response.

30. The method of claim 29 wherein the compound is effective to increase FoxP3+ regulatory T-cells.
